(19) United States
(12) Patent Application Publication
(10) Pub. No.: US 2019/0338302 A1
(43) Pub. Date: Nov. 7, 2019

(54) TRANSGENIC LAND PLANTS COMPRISING A PUTATIVE TRANSPORTER PROTEIN OF AN EDIBLE EUKARYOTIC ALGAE

(71) Applicant: Yield10 Bioscience, Inc., Woburn, MA (US)

(72) Inventors: Oliver P. Peoples, Arlington, MA (US); Kristi D. Snell, Belmont, MA (US); Meghna Malik, SASKATOON (CA)

(73) Assignee: Yield10 Bioscience, Inc., Woburn, MA (US)

(21) Appl. No.: 16/074,812

(22) PCT Filed: Feb. 3, 2017
(86) PCT No.: PCT/US2017/016421

§ 371 Date: Aug. 2, 2018

Related U.S. Application Data
(60) Provisional application No. 62/291,341, filed on Feb. 4, 2016.

(51) Int. Cl. C12N 15/82; C07K 14/405

(52) U.S. Cl. CPC C12N 15/8261; C07K 14/405

(57) ABSTRACT

A transgenic land plant is provided. The transgenic land plant comprises a putative bicarbonate transporter protein of an edible eukaryotic algae. The putative bicarbonate transporter protein of the edible eukaryotic algae is heterologous with respect to the transgenic land plant. The putative bicarbonate transporter protein is an ortholog of CCP1 of Chlamydomonas reinhardtii of SEQ ID NO: 1. The putative bicarbonate transporter protein is localized to mitochondria of the transgenic land plant based on a mitochondrial targeting signal intrinsic to the putative bicarbonate transporter protein.

### FIELD OF THE INVENTION

The present invention relates generally to transgenic land plants, and more particularly, to transgenic land plants comprising a putative bicarbonate transporter protein of an edible eukaryotic algae.

### BACKGROUND OF THE INVENTION

The world faces a major challenge in the next 35 years to meet the increased demands for food production to feed a growing global population, which is expected to reach 9 billion by the year 2050. Food output will need to be increased by up to 70% in view of the growing population. Increased demand for improved diet, concomitant land use changes for new living space and infrastructure, alternative uses for crops such as biofuels and biobased products, and changing weather patterns will add to the challenge.

Major agricultural crops include food crops, such as maize, wheat, oat, barley, soybean, millet, sorghum, potato, pulse, bean, tomato, rice, cassava, sugar beets, and potatoes, among others, forage crop plants, such as hay, alfalfa, and silage corn, among others, and oilseed crops, such as camelina, Brassica species (e.g. B. napus (canola), B. rapa, B. juncea, and B. carinata), crambe, soybean, sunflower, safflower, oil palm, flax, and cotton, among others. Productivity of these crops, and others, is limited by numerous factors, including for example relative inefficiency of photochemical conversion of light energy to fixed carbon during photosynthesis, as well as loss of fixed carbon by photorespiration and/or other metabolic pathways having enzymes catalyzing decarboxylation reactions. Current crop production relies primarily on crop species that were bred by conventional means for improved seed yield which was improved by continuous incremental changes over many years. Over this period any step changes in yield were typically enabled by new technologies such as the advent of nitrogen fertilizers, dwarf wheat varieties, dwarf rice, hybrids such as corn with “hybrid vigor,” and more recently, improved agronomic practices such as increased density of seed planting enabled largely by transgenic input traits including herbicide resistance and pesticide resistance. Unfortunately, given the inherent complexity of plant metabolism and the fact that plants have evolved to balance inputs with growth and reproduction, it is likely that achieving further step changes in crop yield will require new approaches.

One potential approach involves metabolic engineering of crop plants to express carbon-concentrating mechanisms of cyanobacteria or eukaryotic algae. Cyanobacteria and eukaryotic algae have evolved carbon-concentrating mechanisms to increase intracellular concentrations of dissolved inorganic carbon, particularly to increase concentrations of CO₂ at the active site of ribulose-1,5-bisphosphate carboxylase/oxygenase (also termed RubisCO). RubisCO catalyzes carboxylation of one molecule of ribulose-1,5-bisphosphate to yield two molecules of 3-phosphoglycerate, providing the first stable intermediate in C₃ photosynthesis. RubisCO also catalyzes the oxygenation of ribulose-1,5-bisphosphate by a side-reaction, though, to yield one molecule of 3-phosphoglycerate and one molecule of 2-phosphoglycolate, the latter of which is converted to 3-phosphoglycerate via photorespiration, resulting in a substantial increase in the energy required for fixation of carbon and thus a substantial decrease in efficiency. The ratio of carboxylation to oxygenation catalyzed by RubisCO depends, among other things, on the concentrations of CO₂ and O₂ in the vicinity of RubisCO, with high CO₂ concentration promoting carboxylation over oxygenation. The carbon-concentrating mechanism of cyanobacteria include expression of bicarbonate transporters and carbonic anhydrases, to increase CO₂ concentration in the vicinity of RubisCO, and localization of RubisCO in carboxysomes, resulting in up to an approximately 1000-fold increase in CO₂ concentration in the vicinity of RubisCO relative to ambient conditions. Similarly, the carbon-concentrating mechanism of eukaryotic algae includes expression of transporters of inorganic carbon (also termed Ci transporters), including bicarbonate transporters, and α and β carbonic anhydrases for concentration of bicarbonate in chloroplast stroma, along with localization of RubisCO in pyrenoids, resulting in an approximately 40-fold increase in CO₂ concentration in the vicinity of RubisCO relative to ambient conditions. These carbon-concentrating mechanisms result in photosynthetic fixation of carbon with increased efficiency in cyanobacteria and eukaryotic algae. It has been proposed that metabolic engineering of crop plants to express components of these carbon-concentrating mechanisms, individually or in various combinations, may also result in increased photosynthetic efficiency, and thus increased yield, in the crop plants too.

For example, Schnell et al., WO 2015/103074, have reported that expression in transgenic plants of a heterologous putative bicarbonate transporter, e.g., from cyanobacteria or algae, that localizes to chloroplast envelope membranes, leads to significant improvements in levels of photosynthesis in the transgenic plants. Schnell et al., WO 2015/103074, disclose that the putative bicarbonate transporter protein CCP1 of the eukaryotic algae Chlamydomonas reinhardtii, among others, can be used. Schnell et al., WO 2015/103074, also disclose that functional data suggest that Camelina plants transformed to express CCP1 of Chlamydomonas reinhardtii respond to higher CO₂ transport capacity by decreasing transpiration and gas exchange (i.e. closing stomata), and that in field studies involving comparison of plants of three transformants versus wild-type plants, two of the three transformants exhibited an increase in overall oil yield (lb./acre) of 43% and 76%, respectively. Schnell et al., WO 2015/103074, does not appear to suggest that CCP1 may actually localize to mitochondria in addition to, or instead of, localizing to chloroplasts, though, and does not otherwise suggest specific benefits, or differences, associated with use of CCP1 or homologs thereof, relative to other putative bicarbonate transporters.

Also for example, Atkinson et al. (2015), Plant Biotechnol. J., doi: 10.1111/pbi.12497, have reported that intracellular locations of ten components of the carbon-concentrating mechanism of Chlamydomonas reinhardtii were confirmed, and that when expressed in tobacco, all of these components except chloroplastic carbonic anhydrases CAH3 and CAH6 had the same intracellular locations as in Chlamydomonas reinhardtii. Atkinson et al. (2015) discloses that CCP1 and its homolog CCP2, which are characterized as putative Ci transporters previously reported to be in the chloroplast envelope, localized to mitochondria in both Chlamydomonas reinhardtii, as expressed naturally, and tobacco, when expressed heterologously, suggesting that the model for the carbon-concentrating mechanism of eukaryotic algae needs to be expanded to include a role for mitochondria. Atkinson et al. (2015) also discloses that expression of individual putative Ci transporters did not enhance Arabidopsis growth, though, and suggests that stacking of further components of carbon-concentrating mechanisms will probably be required to achieve a significant increase in photosynthetic efficiency in this species, albeit without having tested expression of CCP1 in particular. Atkinson et al. (2015) also does not otherwise suggest specific benefits, or differences, associated with use of CCP1 or homologs thereof, relative to other putative bicarbonate transporters.

Thus, there is a need for transgenic plants with enhanced carbon capture systems to improve crop yield and/or seed yield.

### BRIEF SUMMARY OF THE INVENTION

In accordance with one aspect of the present invention, a transgenic land plant is disclosed. The transgenic land plant comprises a putative bicarbonate transporter protein of an edible eukaryotic algae. The putative bicarbonate transporter protein of the edible eukaryotic algae is heterologous with respect to the transgenic land plant. The putative bicarbonate transporter protein is an ortholog of CCP1 of Chlamydomonas reinhardtii of SEQ ID NO: 1. The putative bicarbonate transporter protein is localized to mitochondria of the transgenic land plant based on a mitochondrial targeting signal intrinsic to the putative bicarbonate transporter protein.

### BRIEF DESCRIPTION OF THE DRAWINGS

FIG. 1 shows predicted transmembrane regions (grey shading) of CCP1 protein of Chlamydomonas reinhardtii of SEQ ID NO: 1, based on Phobius prediction. Data correspond to plots of posterior label probability (y-axis) versus amino acid number of the protein (x-axis), including predicted transmembrane regions (grey shading), cytoplasmic regions (Xs on grey line), non-cytoplasmic regions (filled circles on black line), and signal peptides (open triangles on grey line).

FIG. 2 shows predicted transmembrane regions (grey shading) of a protein of Chlorella sorokiniana (GAPD01006726.1) of SEQ ID NO: 2 that is an ortholog of CCP1, based on Phobius prediction. Data correspond to plots of posterior label probability (y-axis) versus amino acid number of the protein (x-axis), including predicted transmembrane regions (grey shading), cytoplasmic regions (Xs on grey line), non-cytoplasmic regions (filled circles on black line), and signal peptides (open triangles on grey line).

FIG. 3 shows predicted transmembrane regions (grey shading) of a protein of Chlorella variabilis (XM_005846489.1) of SEQ ID NO: 6 that is an ortholog of CCP1, based on Phobius prediction. Data correspond to plots of posterior label probability (y-axis) versus amino acid number of the protein (x-axis), including predicted transmembrane regions (grey shading), cytoplasmic regions (Xs on grey line), non-cytoplasmic regions (filled circles on black line), and signal peptides (open triangles on grey line).

FIG. 4 shows predicted transmembrane regions (grey shading) of a protein of Chlorella variabilis (XM_005852157.1) of SEQ ID NO: 4 that is an ortholog of CCP1, based on Phobius prediction. Data correspond to plots of posterior label probability (y-axis) versus amino acid number of the protein (x-axis), including predicted transmembrane regions (grey shading), cytoplasmic regions (Xs on grey line), non-cytoplasmic regions (filled circles on black line), and signal peptides (open triangles on grey line).

FIG. 5 shows predicted transmembrane regions (grey shading) of a protein of Chlorella variabilis XM_005843001.1 of SEQ ID NO: 5 that is an ortholog of CCP1, based on Phobius prediction. Data correspond to plots of posterior label probability (y-axis) versus amino acid number of the protein (x-axis), including predicted transmembrane regions (grey shading), cytoplasmic regions (Xs on grey line), non-cytoplasmic regions (filled circles on black line), and signal peptides (open triangles on grey line).

FIGS. 6A-B shows a multiple sequence alignment of CCP1 of Chlamydomonas reinhardtii and eight orthologs of CCP1 of eukaryotic edible algae, according to CLUSTAL O(1.2.1), with the sequences presented in the following order: Chlorella_variabilis_XM_005851446 (SEQ ID NO: 3); Chondrus crispus_XM_005712871; (SEQ ID NO: 7); Chlorella_variabilis_XM_005852157.1 (SEQ ID NO: 4); Chlorella_variabilis_XM_005843001 (SEQ ID NO: 5); Chlamydomonas_reinhardtii_CCP1 (SEQ ID NO: 1); Chlorella_sorokiniana_GAPD01006726.1 (SEQ ID NO: 2); Chlorella_variabilis_XM_005846489.1 (SEQ ID NO: 6); Chondrus_crispus_XM_005715654 (SEQ ID NO: 8); and Chondrus_crispus_XM_005713259 (SEQ ID NO: 9).

FIG. 7A-B shows plasmid maps of transformation vectors (A) pMBXO85 and (B) pMBXO86. Plasmid pMBXO85 contains a constitutive expression cassette, containing a CaMV35S promoter operably linked to the 5′ end of an ortholog of the CCP1 gene from an edible algae Chlorella sorokiniana (Nucleotide accession number GAPD01006726.1). The CCP1 ortholog from C. sorokiniana is operably linked at its 3′ end to a CaMV35S polyadenylation sequence. An expression cassette for the bar gene, imparting transgenic plants resistance to the herbicide bialophos, contains the CaMV35S promoter operably linked to the 5′ end of the bar gene. The bar gene is operably linked at the 3′ end to a CaMV35S polyadenylation sequence. Plasmid pMBXO86 contains a constitutive expression cassette, containing a CaMV35S promoter operably linked to the 5′ end of an ortholog of the CCP1 gene from an edible algae Chlorella variabilis (Nucleotide accession number XM_005846489.1). The CCP1 ortholog from C. variabilis is operably linked at its 3′ end to a CaMV35S polyadenylation sequence. An expression cassette for the bar gene, driven by the CaMV35S promoter at the 5′ end of the gene and flanked at the 3′ end by a CaMV35S polyadenylation sequence, imparts transgenic plants resistance to the herbicide bialophos.

FIG. 8 shows a model for further enhanced yield based on inhibiting expression of cell wall invertase inhibitor that would otherwise be upregulated in CCP1 lines.

### DETAILED DESCRIPTION OF THE INVENTION

A transgenic land plant is disclosed. The transgenic land plant comprises a putative bicarbonate transporter protein of an edible eukaryotic algae. The putative bicarbonate transporter protein of the edible eukaryotic algae is heterologous with respect to the transgenic land plant. The putative bicarbonate transporter protein is an ortholog of CCP1 of Chlamydomonas reinhardtii of SEQ ID NO: 1. The putative bicarbonate transporter protein is localized to mitochondria of the transgenic land plant based on a mitochondrial targeting signal intrinsic to the putative bicarbonate transporter protein.

Without wishing to be bound by theory, it is believed that modifying a land plant to express a putative bicarbonate transporter protein of an edible eukaryotic algae to obtain a transgenic land plant, wherein the putative bicarbonate transporter protein of the edible eukaryotic algae is heterologous with respect to the transgenic land plant, is an ortholog of CCP1 of Chlamydomonas reinhardtii of SEQ ID NO: 1, and is localized to mitochondria of the transgenic land plant based on a mitochondrial targeting signal intrinsic to the putative bicarbonate transporter protein, will result in enhanced yield, based for example on an increased CO₂ assimilation rate and/or a decreased transpiration rate of the transgenic land plant, in comparison to a reference land plant not expressing the putative bicarbonate transporter protein. It is believed that the putative bicarbonate transporter protein potentially enhances transport of bicarbonate from or into the mitochondria and/or otherwise alters mitochondrial metabolism by transport of bicarbonate and/or other small molecules, thereby enhancing rates of carbon fixation by increasing CO₂ recovery from photorespiration and respiration. Alternatively, the increased transport of small molecules may prevent the accumulation of photorespiratory intermediates that may inhibit photosynthesis. Moreover, it is believed that by modifying the land plant to express a putative bicarbonate transporter protein that is localized to mitochondria in particular, it will be possible to stack expression of the putative bicarbonate transporter protein with expression of other proteins in deliberate and complementary approaches to further enhance yield. In addition, it is believed that by modifying the land plant to express a putative bicarbonate transporter protein of an edible eukaryotic algae in particular, it will be possible to generate transgenic crops that include only proteins that are already generally recognized as safe for human consumption.

As noted, a transgenic land plant is disclosed. A land plant is a plant belonging to the plant subkingdom Embryophyta.

The term “land plant” includes mature plants, seeds, shoots and seedlings, and parts, propagation material, plant organ tissue, protoplasts, callus and other cultures, for example cell cultures, derived from plants belonging to the plant subkingdom Embryophyta, and all other species of groups of plant cells giving functional or structural units, also belonging to the plant subkingdom Embryophyta. The term “mature plants” refers to plants at any developmental stage beyond the seedling. The term “seedlings” refers to young, immature plants at an early developmental stage.

Land plants encompass all annual and perennial monocotyledonous or dicotyledonous plants and includes by way of example, but not by limitation, those of the genera Cucurbita, Rosa, Vitis, Juglans, Fragaria, Lotus, Medicago, Onobrychis, Trifolium, Trigonella, Vigna, Citrus, Linum, Geranium, Manihot, Daucus, Arabidopsis, Brassica, Raphanus, Sinapis, Atropa, Capsicum, Datura, Hyoscyamus, Lycopersicon, Nicotiana, Solarium, Petunia, Digitalis, Majorana, Cichorium, Helianthus, Lactuca, Bromus, Asparagus, Antirrhinum, Heterocallis, Nemesis, Pelargonium, Panieum, Pennisetum, Ranunculus, Senecio, Salpiglossis, Cucumis, Browaalia, Glycine, Pisum, Phaseolus, Lolium, Oryza, Zea, Avena, Hordeum, Secale, Triticum, Sorghum, Picea, Populus, Camelina, Beta, Solanum, and Carthamus. Preferred land plants are those from the following plant families: Amaranthaceae, Asteraceae, Brassicaceae, Carophyllaceae, Chenopodiaceae, Compositae, Cruciferae, Cucurbitaceae, Euphorbiaceae, Fabaceae, Labiatae, Leguminosae, Papilionoideae, Liliaceae, Linaceae, Malvaceae, Poaceae, Rosaceae, Rubiaceae, Saxifragaceae, Scrophulariaceae, Solanaceae, Sterculiaceae, Tetragoniaceae, Theaceae, Umbelliferae.

The land plant can be a monocotyledonous land plant or a dicotyledonous land plant. Preferred dicotyledonous plants are selected in particular from the dicotyledonous crop plants such as, for example, Asteraceae such as sunflower, tagetes or calendula and others; Compositae, especially the genus Lactuca, very particularly the species sativa (lettuce) and others; Cruciferae, particularly the genus Brassica, very particularly the species napus (oilseed rape), campestris (beet), oleracea cv Tastie (cabbage), oleracea cv Snowball Y (cauliflower) and oleracea cv Emperor (broccoli) and other cabbages; and the genus Arabidopsis, very particularly the species thaliana, and cress or canola and others; Cucurbitaceae such as melon, pumpkin/squash or zucchini and others; Leguminosae, particularly the genus Glycine, very particularly the species max (soybean), soya, and alfalfa, pea, beans or peanut and others; Rubiaceae, preferably the subclass Lamiidae such as, for example Coffea arabica or Coffea liberica (coffee bush) and others; Solanaceae, particularly the genus Lycopersicon, very particularly the species esculentum (tomato), the genus Solanum, very particularly the species tuberosum (potato) and melongena (aubergine) and the genus Capsicum, very particularly the genus annuum (pepper) and tobacco or paprika and others; Sterculiaceae, preferably the subclass Dilleniidae such as, for example, Theobroma cacao (cacao bush) and others; Theaceae, preferably the subclass Dilleniidae such as, for example, Camellia sinensis or Thea sinensis (tea shrub) and others; Umbelliferae, particularly the genus Daucus (very particularly the species carota (carrot)) and Apium (very particularly the species graveolens dulce (celery)) and others; and linseed, cotton, hemp, flax, cucumber, spinach, carrot, sugar beet and the various tree, nut and grapevine species, in particular banana and kiwi fruit. Preferred monocotyledonous plants include maize, rice, wheat, sugarcane, sorghum, oats and barley.

Of particular interest are oilseed plants. In oilseed plants of interest the oil is accumulated in the seed and can account for greater than 10%, greater than 15%, greater than 18%, greater than 25%, greater than 35%, greater than 50% by weight of the weight of dry seed. Oil crops encompass by way of example: Borago officinalis (borage); Camelina (false flax); Brassica species such as B. campestris, B. napus, B. rapa, B. carinata (mustard, oilseed rape or turnip rape); Cannabis sativa (hemp); Carthamus tinctorius (safflower); Cocos nucifera (coconut); Crambe abyssinica (crambe); Cuphea species (Cuphea species yield fatty acids of medium chain length, in particular for industrial applications); Elaeis guinensis (African oil palm); Elaeis oleifera (American oil palm); Glycine max (soybean); Gossypium hirsutum (American cotton); Gossypium barbadense (Egyptian cotton); Gossypium herbaceum (Asian cotton); Helianthus annuus (sunflower); Jatropha curcas (jatropha); Linum usitatissimum (linseed or flax); Oenothera biennis (evening primrose); Olea europaea (olive); Oryza sativa (rice); Ricinus communis (castor); Sesamum indicum (sesame); Thlaspi caerulescens (pennycress); Triticum species (wheat); Zea mays (maize), and various nut species such as, for example, walnut or almond.

Camelina species, commonly known as false flax, are native to Mediterranean regions of Europe and Asia and seem to be particularly adapted to cold semiarid climate zones (steppes and prairies). The species Camelina sativa was historically cultivated as an oilseed crop to produce vegetable oil and animal feed. It has been introduced to the high plain regions of Canada and parts of the United States as an industrial oilseed crop. In addition to being useful as an industrial oilseed crop, Camelina is a very useful model system for developing new tools and transgenic approaches to enhancing the yield of crops in general and for enhancing the yield of seed and seed oil in particular. Demonstrated transgene improvements in Camelina can then be deployed in major oilseed crops including Brassica species including B. napus (canola), B. rapa, B. juncea, B. carinata, crambe, soybean, sunflower, safflower, oil palm, flax, and cotton.

As will be apparent, the land plant can be a C3 plant, i.e. a plant in which RubisCO catalyzes carboxylation of ribulose-1,5-bisphosphate by use of CO₂ drawn directly from the atmosphere, such as for example, wheat, oat, and barley, among others. The land plant also can be a C4 plant, i.e. a plant in which RubisCO catalyzes carboxylation of ribulose-1,5-bisphosphate by use of CO₂ shuttled via malate or aspartate from mesophyll cells to bundle sheath cells, such as for example maize, millet, and sorghum, among others.

Accordingly, in some example the transgenic land plant is a C3 plant. Also, in some examples the transgenic land plant is a C4 plant. Also, in some examples the transgenic land plant is a food crop plant selected from the group consisting of maize, wheat, oat, barley, soybean, millet, sorghum, potato, pulse, bean, tomato, and rice. Also, in some examples the transgenic land plant is a forage crop plant selected from the group consisting of hay, alfalfa, and silage corn. Also, in some examples the transgenic land plant is an oilseed crop plant selected from the group consisting of camelina, Brassica species (e.g. B. napus (canola), B. rapa, B. juncea, and B. carinata), crambe, soybean, sunflower, safflower, oil palm, flax, and cotton.

The transgenic land plant comprises a putative bicarbonate transporter protein of an edible eukaryotic algae. As noted above, it is believed that the putative bicarbonate transporter protein potentially enhances transport of bicarbonate from or into the mitochondria and/or otherwise alters mitochondrial metabolism by transport of bicarbonate and/or other small molecules. Accordingly, the putative bicarbonate transporter protein may be a protein that transports bicarbonate by any transport mechanism. Classes of bicarbonate transport proteins include anion exchangers and Na⁺/HCO₃⁻¹ symporters. The putative bicarbonate transporter protein also may be a protein that otherwise alters mitochondrial metabolism by transport of bicarbonate and/or other small molecules. Increased transport of other small molecules may prevent their buildup which might otherwise inhibit photosynthesis.

As noted, the transgenic land plant comprises a putative bicarbonate transporter protein of an edible eukaryotic algae. An edible eukaryotic algae is a plant, ranging from a microscopic unicellular form, e.g. a single-cell algae, to a macroscopic multicellular form, e.g. a seaweed, that includes chlorophyll a and, if multicellular, a thallus not differentiated into roots, stem, and leaves, that is classified as chlorophyta (also termed green algae), rhodophyta (also termed red algae), or phaeophyta (also termed brown algae), and that is generally recognized as a typical and suitable component of a human diet. Edible eukaryotic algae include, for example, single-cell algae, including the chlorophyta Chlorella sorokiniana and Chlorella variabdis. Edible eukaryotic algae also include, for example, seaweed, including the chlorophyta Ulva lactuca (also termed sea lettuce) and Enteromorpha (Ulva) intenstinalis (also termed sea grass), the rhodophyta Chondrus crispus (also termed Irish moss or carrigeen), Porphyra umbilicalis (also termed nori), and Palmaria palmata (also termed dulse or dillisk), and the phaeophyta Ascophyllum nodosum (also termed egg wrack), Laminaria digitata (also termed kombu/konbu), Laminaria saccharina (also termed royal or sweet kombu), Himanthalia elongata (also termed sea spaghetti), and Undaria pinnatifida (also termed wakame).

Thus, for example, the putative bicarbonate transporter protein of the edible eukaryotic algae can be a putative bicarbonate transporter protein of a chlorophyta, a putative bicarbonate transporter protein of a rhodophyta, or a putative bicarbonate transporter protein of a phaeophyta. Also for example, the putative bicarbonate transporter protein of the edible eukaryotic algae can be a putative bicarbonate transporter protein of a single-cell algae or a putative bicarbonate transporter protein of a seaweed. Also for example, the putative bicarbonate transporter protein of the edible eukaryotic algae can be a putative bicarbonate transporter protein of Chlorella sorokiniana or a putative bicarbonate transporter protein of Chlorella variabilis, among others. Also for example, the putative bicarbonate transporter protein of the edible eukaryotic algae can be a putative bicarbonate transporter protein of Ulva lactuca, a putative bicarbonate transporter protein of Enteromorpha (Ulva) intenstinalis, a putative bicarbonate transporter protein of Chondrus crispus, a putative bicarbonate transporter protein of Porphyra umbilicalis, a putative bicarbonate transporter protein of Palmaria palmata, a putative bicarbonate transporter protein of Ascophyllum nodosum, a putative bicarbonate transporter protein of Laminaria digitata, a putative bicarbonate transporter protein of Laminaria saccharina, a putative bicarbonate transporter protein of Himanthalia elongata, or a putative bicarbonate transporter protein of Undaria pinnatifida, among others.

In contrast, Chlamydomonas reinhardtii is not an edible eukaryotic algae. Chlamydomonas reinhardtii is a single-cell eukaryotic algae corresponding to a chlorophyta, but Chlamydomonas reinhardtii is not generally recognized as a typical and suitable component of a human diet, and thus is not an edible eukaryotic algae. Accordingly, the putative bicarbonate transporter protein of the edible eukaryotic algae is not a putative bicarbonate transporter protein of Chlamydomonas reinhardtii.

The putative bicarbonate transporter protein of the edible eukaryotic algae is heterologous with respect to the transgenic land plant. By this it is meant that the putative bicarbonate transporter protein of the edible eukaryotic algae is not normally expressed or otherwise present in land plants of the type from which the transgenic land plant is derived, i.e. land plants of the type from which the transgenic land plant is derived do not express any protein having an amino acid sequence identical to that of the putative bicarbonate transporter protein of the edible eukaryotic algae. Rather, the transgenic land plant comprises the putative bicarbonate transporter protein of the edible eukaryotic algae based on modification of a land plant to express the putative bicarbonate transporter protein of the edible eukaryotic algae, thus resulting in the transgenic land plant.

The putative bicarbonate transporter protein is an ortholog of CCP1 of Chlamydomonas reinhardtii of SEQ ID NO: 1. The term “ortholog” means a polynucleotide sequence or polypeptide sequence possessing a high degree of homology, i.e. sequence relatedness, to a subject sequence and being a functional equivalent of the subject sequence, wherein the sequence that is orthologous is from a species that is different than that of the subject sequence. Homology may be quantified by determining the degree of identity and/or similarity between the sequences being compared.

As used herein, “percent homology” of two polynucleotide sequences or of two polypeptide sequences is determined using the algorithm of Karlin and Altschul (1990), Proc. Natl. Acad. Sci., U.S.A. 87: 2264-2268. Such an algorithm is incorporated into the NBLAST and XBLAST programs of Altschul et al. (1990), J. Mol. Biol. 215: 403-410. BLAST nucleotide searches are performed with the NBLAST program, score=100, word length 12, to obtain nucleotide sequences homologous to a reference polynucleotide sequence. BLAST protein searches are performed with the XBLAST program, score=50, word length=3, to obtain amino acid sequences homologous to a reference polypeptide sequence. To obtain gapped alignments for comparison purposes, Gapped BLAST is utilized as described in Altschul et al. (1997), Nucleic Acids Res. 25: 3389-3402. When utilizing BLAST and Gapped BLAST programs, the default parameters are typically used.

In the case of polypeptide sequences that are less than 100% identical to a reference sequence, the non-identical positions are preferably, but not necessarily, conservative substitutions for the reference sequence. Conservative substitutions typically include substitutions within the following groups: glycine and alanine; valine, isoleucine, and leucine; aspartic acid and glutamic acid; asparagine and glutamine; serine and threonine; lysine and arginine; and phenylalanine and tyrosine.

Where a particular polypeptide is said to have a specific percent identity to a reference polypeptide of a defined length, the percent identity is relative to the reference peptide. Thus, a peptide that is 50% identical to a reference polypeptide that is 100 amino acids long can be a 50 amino acid polypeptide that is completely identical to a 50 amino acid long portion of the reference polypeptide. It might also be a 100 amino acid long polypeptide that is 50% identical to the reference polypeptide over its entire length. Many other polypeptides will meet the same criteria.

For reference, as discussed above CCP1 is a putative bicarbonate transporter of Chlamydomonas reinhardtii. In addition, CCP1 has an amino acid sequence in accordance with SEQ ID NO: 1.

Accordingly, the putative bicarbonate transporter protein is a polypeptide sequence possessing a high degree of sequence relatedness to CCP1 of Chlamydomonas reinhardtii of SEQ ID NO: 1 and being a functional equivalent thereof, wherein the putative bicarbonate transporter protein is from a species that is different than Chlamydomonas reinhardtii.

The putative bicarbonate transporter protein is localized to mitochondria of the transgenic land plant based on a mitochondrial targeting signal intrinsic to the putative bicarbonate transporter protein. The putative bicarbonate transporter protein can be localized to mitochondria for example based on being encoded by DNA present in the nucleus of a plant cell, synthesized in the cytosol of the plant cell, targeted to the mitochondria of the plant cell, and inserted into outer membranes and/or inner membranes of the mitochondria. A mitochondrial targeting signal is a portion of a polypeptide sequence that targets the polypeptide sequence to mitochondria. A mitochondrial targeting signal intrinsic to the putative bicarbonate transporter protein is a mitochondrial targeting signal that is integral to the putative bicarbonate transporter protein, e.g. based on occurring naturally at the N-terminal end of the putative bicarbonate transporter protein or in discrete segments along the putative bicarbonate transporter protein. This is in contrast, for example, to fusion of a heterologous mitochondrial targeting signal to a putative bicarbonate transporter protein that would not otherwise be targeted to mitochondria. For reference, also as discussed above CCP1 is localized to mitochondria in both Chlamydomonas reinhardtii, as expressed naturally, and tobacco, when expressed heterologously. Accordingly, the putative bicarbonate transporter protein can be a putative bicarbonate transporter protein that is encoded by nuclear DNA, synthesized cytosolically, targeted to the mitochondria, and inserted into outer membranes and/or inner membranes thereof, based on targeting by a portion of the polypeptide sequence integral to putative bicarbonate transporter protein. The putative bicarbonate transporter protein does not have typical plastid targeting signals.

Suitable putative bicarbonate transporter proteins can be identified, for example, based on searching databases of polynucleotide sequences or polypeptide sequences for orthologs of CCP1 of Chlamydomonas reinhardtii of SEQ ID NO: 1, wherein the polynucleotide sequences or polypeptide sequences being derived from edible eukaryotic algae. Such searches can be carried out, for example, by use of BLAST, e.g. tblastn, and databases including translated polynucleotides, whole genome shotgun sequences, and/or transcriptome assembly sequences, among other sequences and databases, as discussed above. Potential orthologs of CCP1 may be identified, for example, based on percentage of identity and/or percentage of similarity, with respect to polypeptide sequence, of individual sequences in the databases in comparison to CCP1 of Chlamydomonas reinhardtii, also as discussed above. For example, potential orthologs of CCP1 may be identified based on percentage of identity of an individual sequence in a database and CCP1 of Chlamydomonas reinhardtii of SEQ ID NO: 1 of at least 25%, e.g. at least 30%, at least 35%, at least 40%, at least 45%, at least 50%, at least 55%, at least 60%, at least 65%, at least 70%, at least 75%, at least 80%, at least 90%, or at least 95%, wherein the individual sequence is derived from edible eukaryotic algae. Also for example, potential orthologs of CCP1 may be identified based on percentage of similarity of an individual sequence in a database and CCP1 of Chlamydomonas reinhardtii of SEQ ID NO: 1 of at least 10%, e.g. at least 15%, at least 20%, at least 25%, at least 30%, at least 35%, at least 40%, at least 45%, at least 50%, at least 55%, at least 60%, at least 65%, at least 70%, at least 75%, at least 80%, at least 90%, or at least 95%, wherein the individual sequence is derived from edible eukaryotic algae. Also for example, potential orthologs of CCP1 may be identified based on both percentage of identity of at least 25%, e.g. at least 30%, at least 35%, at least 40%, at least 45%, at least 50%, at least 55%, at least 60%, at least 65%, at least 70%, at least 75%, at least 80%, at least 90%, or at least 95%, and percentage of similarity of at least 10%, e.g. at least 15%, at least 20%, at least 25%, at least 30%, at least 35%, at least 40%, at least 45%, at least 50%, at least 55%, at least 60%, at least 65%, at least 70%, at least 75%, at least 80%, at least 90%, or at least 95%, wherein the individual sequence is derived from edible eukaryotic algae.

Suitable putative bicarbonate transporter proteins also can be identified, for example, based on functional screens.

For example, some cyanobacterial bicarbonate transporters have previously been shown to functionally localize into the E. coli cytoplasmic membrane, including some bicarbonate transporters, as reported by Du et al. (2014), PLoS One 9, e115905. Expression of six particular cyanobacterial bicarbonate transporters in Escherichia coli using a mutant E. coli strain, termed EDCM636, that that is deficient in carbonic anhydrase activity and that is unable to grow on LB or M9 plates without supplementation with high levels of CO₂, restored growth of the E. coli mutant at atmospheric levels of CO₂, whereas expression of various others did not, as reported by Du et al. (2014). Function of CCP1 and potential orthologs thereof with respect to bicarbonate transport may be tested by an analogous approach, and corresponding functional screens developed, also based on restoring growth of this mutant E. coli strain that is deficient in carbonic anhydrase activity based on expressing CCP1 or potential orthologs thereof in the mutant E. coli strain.

Function of CCP1 and potential orthologs thereof with respect to bicarbonate transport also may be tested, and corresponding functional screens developed, based use of yeast modified to express CCP1 and potential orthologs thereof. Transport of bicarbonate from mitochondria of yeast so modified would indicate that these sequences also enable transport of bicarbonate in yeast.

Following identification of a putative bicarbonate transporter protein of an edible eukaryotic algae, modification of a land plant to express the putative bicarbonate transporter protein can be carried out by methods that are known in the art, as discussed in detail below.

The transgenic land plant can be a transgenic land plant wherein the only heterologous algal protein that the transgenic land plant comprises is the putative bicarbonate transporter protein. As noted above, Atkinson et al. (2015) also discloses that expression of individual putative Ci transporters did not enhance Arabidopsis growth, and suggests that stacking of further components of carbon-concentrating mechanisms will probably be required to achieve a significant increase in photosynthetic efficiency in this species, albeit without having tested expression of CCP1 in particular. In contrast, without wishing to be bound by theory, it is believed that a transgenic land plant comprising a putative bicarbonate transporter protein of an edible eukaryotic algae, wherein the putative bicarbonate transporter protein of the edible eukaryotic algae is heterologous with respect to the transgenic land plant, the putative bicarbonate transporter protein is an ortholog of CCP1 of Chlamydomonas reinhardtii of SEQ ID NO: 1, and the putative bicarbonate transporter protein is localized to mitochondria of the transgenic land plant based on a mitochondrial targeting signal intrinsic to the putative bicarbonate transporter protein, will achieve a significant increase in photosynthetic efficiency in the transgenic land plant without need for stacking of further components of carbon-concentrating mechanisms, and thus without expression of any other heterologous algal protein by the transgenic land plant. The corresponding transgenic land plant will provide advantages relative to plants that are modified to express multiple genes, for example in terms of simpler methods of making the transgenic land plant, and lower risk of harmful effects of heterologous proteins with respect to use of the transgenic land plant as a food crop, a forage crop, or an oilseed crop.

Considering the putative bicarbonate transporter protein in more detail, the putative bicarbonate transporter protein can correspond to a putative bicarbonate transporter protein selected from among specific polypeptide sequences of edible eukaryotic algae. As noted above, putative bicarbonate transporter proteins may be identified based on homology to CCP1. Exemplary putative bicarbonate transporter proteins identified this way include a putative bicarbonate transporter protein of an edible Chlorella sorokiniana of SEQ ID NO: 2. Such exemplary putative bicarbonate transporter proteins also include putative bicarbonate transporter proteins of an edible Chlorella variabilis of SEQ ID NO: 3, SEQ ID NO: 4, SEQ ID NO: 5, and SEQ ID NO: 6. Such exemplary putative bicarbonate transporter proteins also include putative bicarbonate transporter proteins of an edible Chondrus crispus of SEQ ID NO: 7, SEQ ID NO: 8, and SEQ ID NO: 9. Thus, for example, the putative bicarbonate transporter protein can correspond to a putative bicarbonate transporter protein selected from the group consisting of (a) a putative bicarbonate transporter protein of an edible Chlorella sorokiniana of SEQ ID NO: 2, (b) a putative bicarbonate transporter protein of an edible Chlorella variabilis of SEQ ID NO: 3, SEQ ID NO: 4, SEQ ID NO: 5, or SEQ ID NO: 6, and (c) a putative bicarbonate transporter protein of an edible Chondrus crispus of SEQ ID NO: 7, SEQ ID NO: 8, or SEQ ID NO: 9.

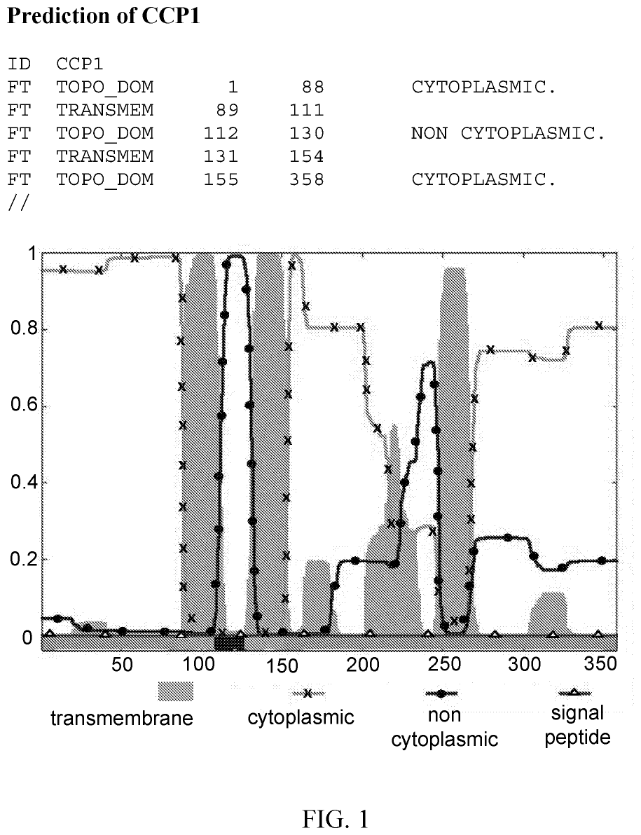

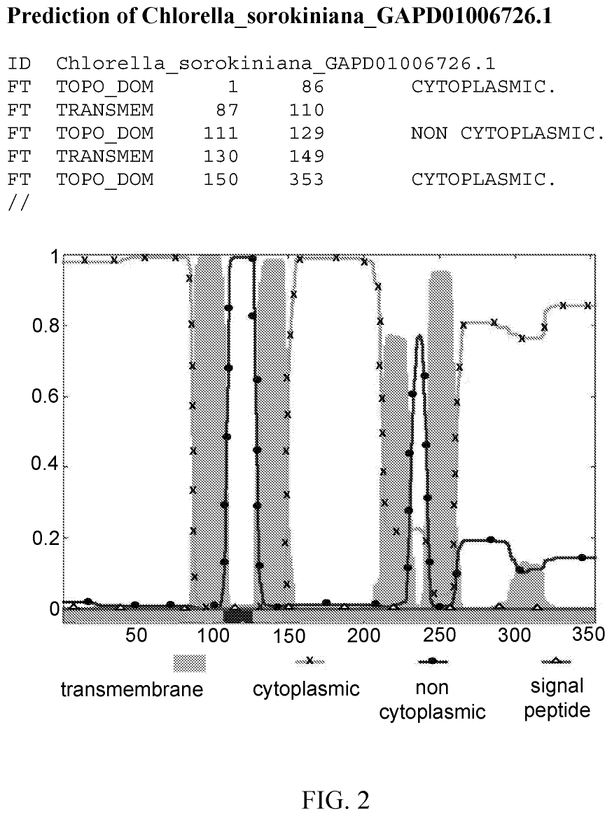

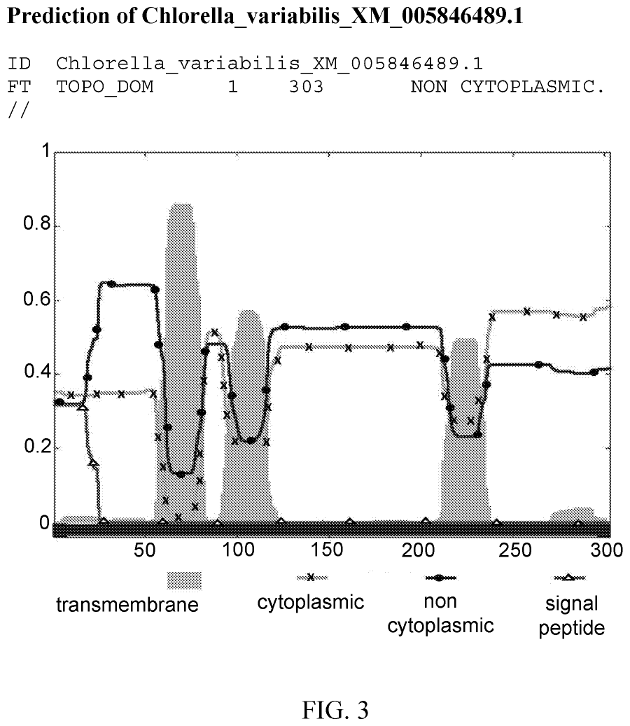

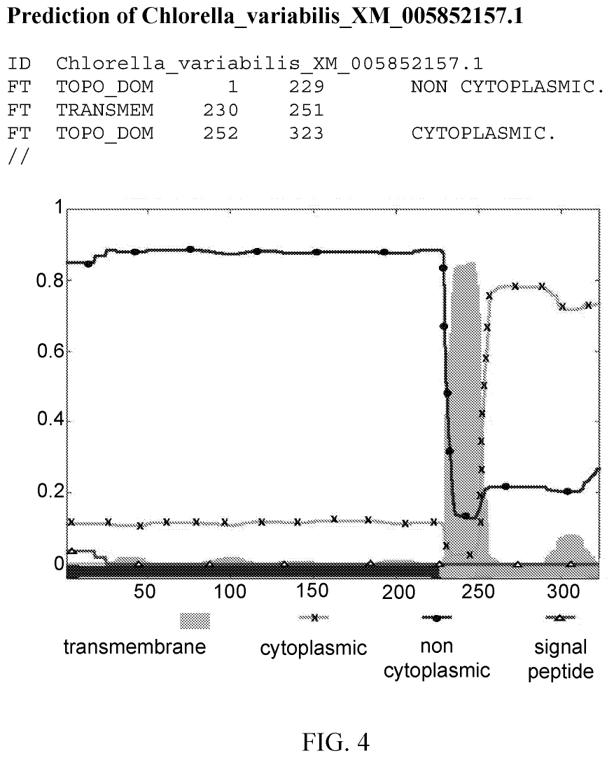

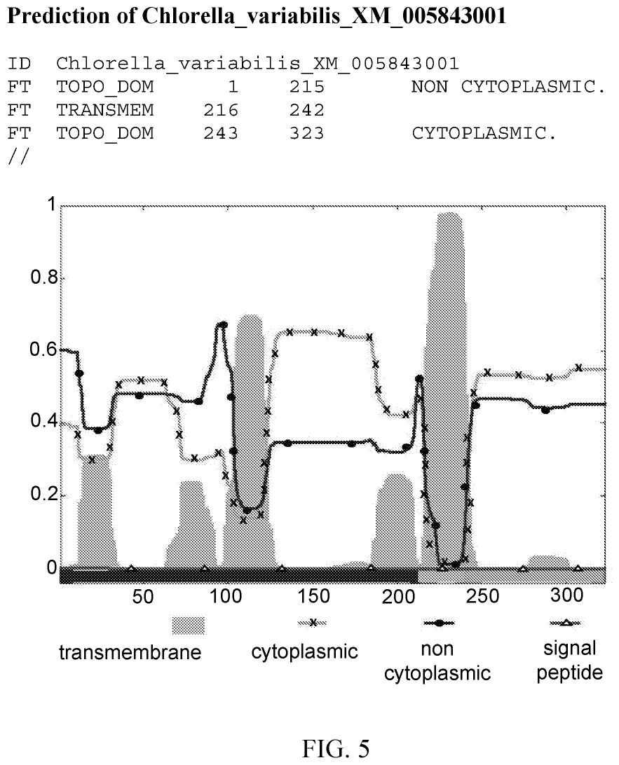

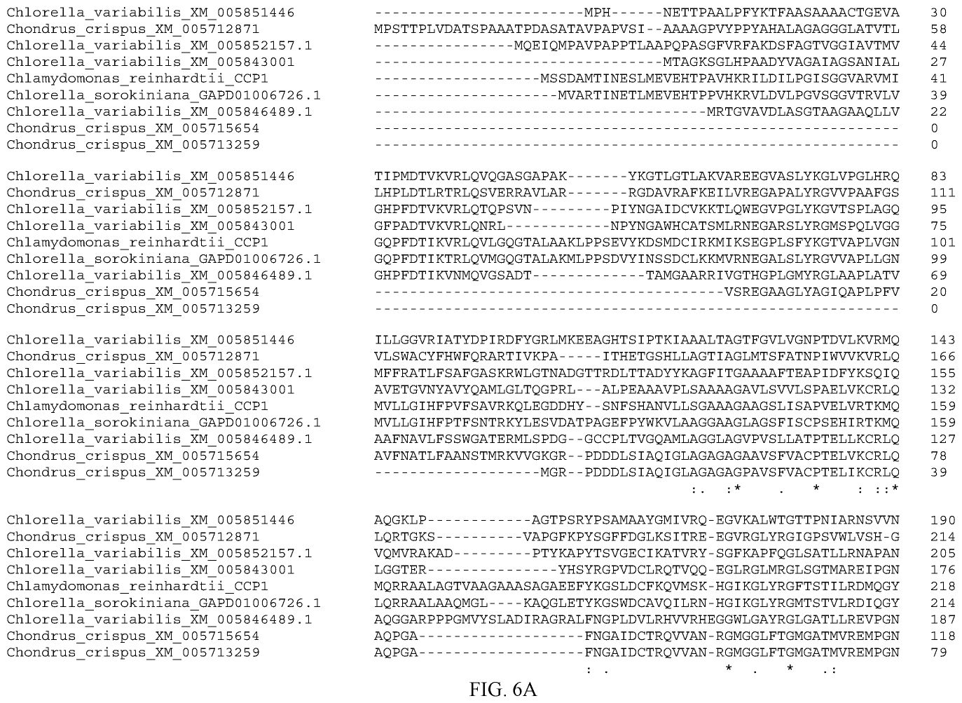

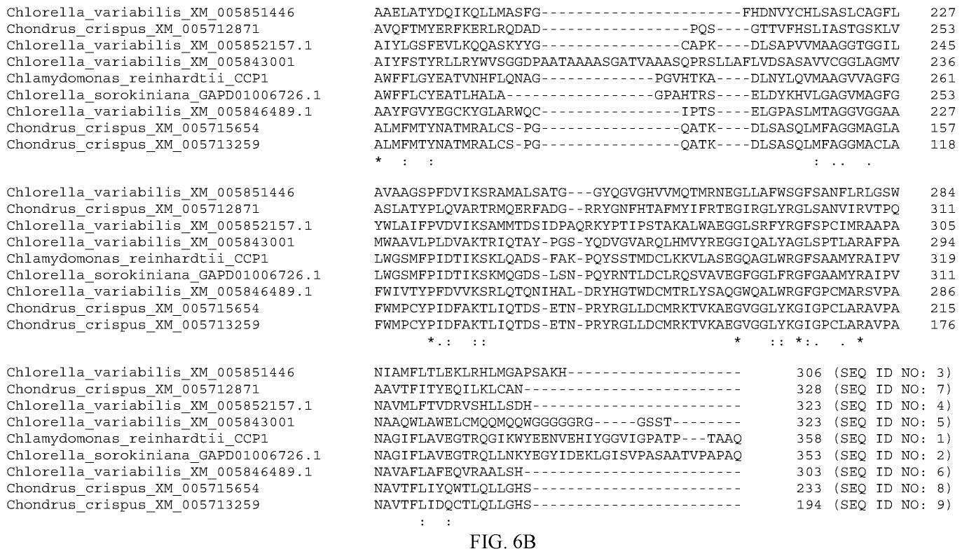

The putative bicarbonate transporter protein also can correspond to a putative bicarbonate transporter protein including specific structural features and characteristics shared among orthologs of CCP1 of Chlamydomonas reinhardtii of SEQ ID NO: 1. With reference to FIG. 1, FIG. 2, FIG. 3, FIG. 4, FIG. 5, and FIGS. 6A-B, such structural features and characteristics shared among the various orthologs of CCP1, namely the putative bicarbonate transporter proteins of SEQ ID NO: 2 to SEQ ID NO: 9, as identified based on multiple sequence alignment of CCP1 and the orthologs, include (i) (a) a proline residue at position 268, (b) an aspartate residue or glutamine residue at position 270, (c) a lysine residue or arginine residue at position 273, and (d) a serine residue or threonine residue at position 274, with numbering of positions relative to CCP1 of Chlamydomonas reinhardtii of SEQ ID NO: 1, and (ii) an overall identity of at least 15%. The noted amino acid residues, i.e. proline residue at position 268, aspartate residue or glutamine residue at position 270, lysine residue or arginine residue at position 273, and serine residue or threonine residue at position 274, with numbering of positions relative to CCP1 of Chlamydomonas reinhardtii of SEQ ID NO: 1, occur at or after the C-terminal portion of a potential transmembrane region of each of CCP1 and at least four of the orthologs, namely that of Chlorella sorokiniana (GAPD01006726.1) of SEQ ID NO: 2, that of Chlorella variabilis (XM_005846489.1) of SEQ ID NO: 6, that of Chlorella variabilis (XM_005852157.1) of SEQ ID NO: 4, and that of Chlorella variabilis XM_005843001.1 of SEQ ID NO: 5. Conservation of the noted amino acid residues, in combination with an overall identity of at least 15%, suggests a structure/function relationship shared among such putative bicarbonate transporter proteins. Thus, for example, the putative bicarbonate transporter protein can be an ortholog of CCP1 of Chlamydomonas reinhardtii of SEQ ID NO: 1 based on comprising: (i) (a) a proline residue at position 268, (b) an aspartate residue or glutamine residue at position 270, (c) a lysine residue or arginine residue at position 273, and (d) a serine residue or threonine residue at position 274, with numbering of positions relative to CCP1 of Chlamydomonas reinhardtii of SEQ ID NO: 1, and (ii) an overall identity of at least 15%.

The putative bicarbonate transporter protein also can correspond to a putative bicarbonate transporter protein that does not only localize to mitochondria, but that also localizes to chloroplasts. As noted above, Atkinson et al. discloses that CCP1 and its homolog CCP2, which are characterized as putative Ci transporters previously reported to be in the chloroplast envelope, localized to mitochondria in both Chlamydomonas reinhardtii, as expressed naturally, and tobacco, when expressed heterologously. Without wishing to be bound by theory, it is believed that localization of CCP1 and orthologs thereof to mitochondria to a greater extent than to chloroplasts promotes enhanced yield. Thus, for example, the putative bicarbonate transporter protein can be localized to mitochondria of the transgenic land plant to a greater extent than to chloroplasts of the transgenic land plant by a factor of at least 2, at least 5, or at least 10.

The putative bicarbonate transporter protein also can correspond to a putative bicarbonate transporter protein that does not differ in any biologically significant way from a wild-type edible eukaryotic algal putative bicarbonate transporter protein. As noted above, the putative bicarbonate transporter protein is localized to mitochondria of the transgenic land plant based on a mitochondrial targeting signal intrinsic to the putative bicarbonate transporter protein, and that this is in contrast, for example, to fusion of a heterologous mitochondrial targeting signal to a putative bicarbonate transporter protein that would not otherwise be targeted to mitochondria. In some examples, the putative bicarbonate transporter protein also does not include any other modifications that might result in the putative bicarbonate transporter protein differing in a biologically significant way from a wild-type edible eukaryotic algal putative bicarbonate transporter protein. Thus, for example the putative bicarbonate transporter protein can consist essentially of an amino acid sequence that is identical to that of a wild-type edible eukaryotic algal putative bicarbonate transporter protein. The corresponding transgenic land plant will provide advantages, e.g. again in terms of lower risk of harmful effects with respect to use of the transgenic land plant as a food crop, a forage crop, or an oilseed crop.

The transgenic land plant can further comprise a heterologous polynucleotide, wherein the putative bicarbonate transporter protein is encoded by the heterologous polynucleotide. For example, the heterologous polynucleotide can comprise a heterologous promoter. Also for example, the heterologous promoter can be a chemical-regulated promoter, a constitutive promoter, or a tissue-preferred promoter. Also for example, the heterologous polynucleotide can be integrated into genomic DNA of the transgenic land plant. These exemplary features of the heterologous polynucleotide, and others, are discussed in detail below.

The transgenic land plant can have a CO₂ assimilation rate that is higher than for a corresponding reference land plant not comprising the putative bicarbonate transporter protein. For example, the transgenic land plant can have a CO₂ assimilation rate that is at least 5% higher, at least 10% higher, at least 20% higher, or at least 40% higher, than for a corresponding reference land plant not comprising the putative bicarbonate transporter protein.

The transgenic land plant also can have a transpiration rate that is lower than for a corresponding reference land plant not comprising the putative bicarbonate transporter protein. For example, the transgenic land plant can have transpiration rate that is at least 5% lower, at least 10% lower, at least 20% lower, or at least 40% lower, than for a corresponding reference land plant not comprising the putative bicarbonate transporter protein.

The transgenic land plant can have a seed yield that is higher than for a corresponding reference land plant not comprising the putative bicarbonate transporter protein. For example, the transgenic land plant can have a seed yield that is at least 5% higher, at least 10% higher, at least 20% higher, at least 40% higher, at least 60% higher, or at least 80% higher, than for a corresponding reference land plant not comprising the putative bicarbonate transporter protein.

As noted above, following identification of a putative bicarbonate transporter protein of an edible eukaryotic algae, modification of a land plant to express the putative bicarbonate transporter protein can be carried out by methods that are known in the art, for example as follows.

DNA constructs useful in the methods described herein include transformation vectors capable of introducing transgenes into land plants. As used herein, “transgenic” refers to an organism in which a nucleic acid fragment containing a heterologous nucleotide sequence has been introduced. The transgenes in the transgenic organism are preferably stable and inheritable. The heterologous nucleic acid fragment may or may not be integrated into the host genome.

Several plant transformation vector options are available, including those described in Gene Transfer to Plants, 1995, Potrykus et al., eds., Springer-Verlag Berlin Heidelberg New York, Transgenic Plants: A Production System for Industrial and Pharmaceutical Proteins, 1996, Owen et al., eds., John Wiley & Sons Ltd. England, and Methods in Plant Molecular Biology: A Laboratory Course Manual, 1995, Maliga et al., eds., Cold Spring Laboratory Press, New York. Plant transformation vectors generally include one or more coding sequences of interest under the transcriptional control of 5′ and 3′ regulatory sequences, including a promoter, a transcription termination and/or polyadenylation signal, and a selectable or screenable marker gene.

Many vectors are available for transformation using Agrobacterium tumefaciens. These typically carry at least one T-DNA sequence and include vectors such as pBIN19. Typical vectors suitable for Agrobacterium transformation include the binary vectors pCIB200 and pCIB2001, as well as the binary vector pCIB 10 and hygromycin selection derivatives thereof (See, for example, U.S. Pat. No. 5,639,949).

Transformation without the use of Agrobacterium tumefaciens circumvents the requirement for T-DNA sequences in the chosen transformation vector and consequently vectors lacking these sequences are utilized in addition to vectors such as the ones described above which contain T-DNA sequences. The choice of vector for transformation techniques that do not rely on Agrobacterium depends largely on the preferred selection for the species being transformed. Typical vectors suitable for non-Agrobacterium transformation include pCIB3064, pSOG 19, and pSOG35. (See, for example, U.S. Pat. No. 5,639,949). Alternatively, DNA fragments containing the transgene and the necessary regulatory elements for expression of the transgene can be excised from a plasmid and delivered to the plant cell using microprojectile bombardment-mediated methods.

Zinc-finger nucleases (ZFNs) are also useful for practicing the invention in that they allow double strand DNA cleavage at specific sites in plant chromosomes such that targeted gene insertion or deletion can be performed (Shukla et al., 2009, Nature 459: 437-441; Townsend et al., 2009, Nature 459: 442-445).

The CRISPR/Cas9 system (Sander, J. D. and Joung, J. K., Nature Biotechnology, published online Mar. 2, 2014; doi;10.1038/nbt.2842) is particularly useful for editing plant genomes to modulate the expression of homologous genes encoding enzymes, for example the NADP-specific malate dehydrogenase enzyme found naturally in the plant cell plastids useful for practicing the disclosed invention. Several examples of the use of this technology to edit the genomes of plants have now been reported (Belhaj et al. Plant Methods 2013, 9:39).

Transformation protocols as well as protocols for introducing nucleotide sequences into plants may vary depending on the type of plant or plant cell targeted for transformation. Suitable methods of introducing nucleotide sequences into plant cells and subsequent insertion into the plant genome include microinjection (Crossway et al. (1986) Biotechniques 4:320-334), electroporation (Riggs et al. (1986) Proc. Natl. Acad. Sci. USA 83:5602-5606), Agrobacterium-mediated transformation (Townsend et al., U.S. Pat. No. 5,563,055; Zhao et al. WO US98/01268), direct gene transfer (Paszkowski et al. (1984) EMBO J. 3:2717-2722), and ballistic particle acceleration (see, for example, Sanford et al., U.S. Pat. No. 4,945,050; Tomes et al. (1995) Plant Cell, Tissue, and Organ Culture: Fundamental Methods, ed. Gamborg and Phillips (Springer-Verlag, Berlin); and McCabe et al. Biotechnology 6:923-926 (1988)). Also see Weissinger et al. Ann. Rev. Genet. 22:421-477 (1988); Sanford et al. Particulate Science and Technology 5:27-37 (1987) (onion); Christou et al. Plant Physiol. 87:671-674 (1988) (soybean); McCabe et al. (1988) BioTechnology 6:923-926 (soybean); Finer and McMullen In Vitro Cell Dev. Biol. 27P:175-182 (1991) (soybean); Singh et al. Theor. Appl. Genet. 96:319-324 (1998)(soybean); Dafta et al. (1990) Biotechnology 8:736-740 (rice); Klein et al. Proc. Natl. Acad. Sci. USA 85:4305-4309 (1988) (maize); Klein et al. Biotechnology 6:559-563 (1988) (maize); Tomes, U.S. Pat. No. 5,240,855; Buising et al., U.S. Pat. Nos. 5,322,783 and 5,324,646; Tomes et al. (1995) in Plant Cell, Tissue, and Organ Culture: Fundamental Methods, ed. Gamborg (Springer-Verlag, Berlin) (maize); Klein et al. Plant Physiol. 91:440-444 (1988) (maize); Fromm et al. Biotechnology 8:833-839 (1990) (maize); Hooykaas-Van Slogteren et al. Nature 311:763-764 (1984); Bowen et al., U.S. Pat. No. 5,736,369 (cereals); Bytebier et al. Proc. Natl. Acad. Sci. USA 84:5345-5349 (1987) (Liliaceae); De Wet et al. in The Experimental Manipulation of Ovule Tissues, ed. Chapman et al. (Longman, N.Y.), pp. 197-209 (1985) (pollen); Kaeppler et al. Plant Cell Reports 9:415-418 (1990) and Kaeppler et al. Theor. Appl. Genet. 84:560-566 (1992) (whisker-mediated transformation); D'Halluin et al. Plant Cell 4:1495-1505 (1992) (electroporation); Li et al. Plant Cell Reports 12:250-255 (1993) and Christou and Ford Annals of Botany 75:407-413 (1995) (rice); Osjoda et al. Nature Biotechnology 14:745-750 (1996) (maize via Agrobacterium tumefaciens); all of which are herein incorporated by reference in their entirety. References for protoplast transformation and/or gene gun for Agrisoma technology are described in WO 2010/037209. Methods for transforming plant protoplasts are available including transformation using polyethylene glycol (PEG), electroporation, and calcium phosphate precipitation (see for example Potrykus et al., 1985, Mol. Gen. Genet., 199, 183-188; Potrykus et al., 1985, Plant Molecular Biology Reporter, 3, 117-128), Methods for plant regeneration from protoplasts have also been described [Evans et al., in Handbook of Plant Cell Culture, Vol 1, (Macmillan Publishing Co., New York, 1983); Vasil, I K in Cell Culture and Somatic Cell Genetics (Academic, Orlando, 1984)].

Recombinase technologies which are useful for producing the disclosed transgenic plants include the cre-lox, FLP/FRT and Gin systems. Methods by which these technologies can be used for the purpose described herein are described for example in (U.S. Pat. No. 5,527,695; Dale and Ow, 1991, Proc. Natl. Acad. Sci. USA 88: 10558-10562; Medberry et al., 1995, Nucleic Acids Res. 23: 485-490).

Transformation protocols as well as protocols for introducing nucleotide sequences into plants may vary depending on the type of plant or plant cell, i.e., monocot or dicot, targeted for transformation.

Suitable methods of introducing nucleotide sequences into plant cells and subsequent insertion into the plant genome are described in US 2010/0229256 A1 to Somleva & Ali and US 2012/0060413 to Somleva et al.

The transformed cells are grown into plants in accordance with conventional techniques. See, for example, McCormick et al., 1986, Plant Cell Rep. 5: 81-84. These plants may then be grown, and either pollinated with the same transformed variety or different varieties, and the resulting hybrid having constitutive expression of the desired phenotypic characteristic identified. Two or more generations may be grown to ensure that constitutive expression of the desired phenotypic characteristic is stably maintained and inherited and then seeds harvested to ensure constitutive expression of the desired phenotypic characteristic has been achieved.

Procedures for in planta transformation can be simple. Tissue culture manipulations and possible somaclonal variations are avoided and only a short time is required to obtain transgenic plants. However, the frequency of transformants in the progeny of such inoculated plants is relatively low and variable. At present, there are very few species that can be routinely transformed in the absence of a tissue culture-based regeneration system. Stable Arabidopsis transformants can be obtained by several in planta methods including vacuum infiltration (Clough & Bent, 1998, The Plant J. 16: 735-743), transformation of germinating seeds (Feldmann & Marks, 1987, Mol. Gen. Genet. 208: 1-9), floral dip (Clough and Bent, 1998, Plant J. 16: 735-743), and floral spray (Chung et al., 2000, Transgenic Res. 9: 471-476). Other plants that have successfully been transformed by in planta methods include rapeseed and radish (vacuum infiltration, Ian and Hong, 2001, Transgenic Res., 10: 363-371; Desfeux et al., 2000, Plant Physiol. 123: 895-904), Medicago truncatula (vacuum infiltration, Trieu et al., 2000, Plant J. 22: 531-541), camelina (floral dip, WO/2009/117555 to Nguyen et al.), and wheat (floral dip, Zale et al., 2009, Plant Cell Rep. 28: 903-913). In planta methods have also been used for transformation of germ cells in maize (pollen, Wang et al. 2001, Acta Botanica Sin., 43, 275-279; Zhang et al., 2005, Euphytica, 144, 11-22; pistils, Chumakov et al. 2006, Russian J. Genetics, 42, 893-897; Mamontova et al. 2010, Russian J. Genetics, 46, 501-504) and Sorghum (pollen, Wang et al. 2007, Biotechnol. Appl. Biochem., 48, 79-83).

Following transformation by any one of the methods described above, the following procedures can be used to obtain a transformed plant expressing the transgenes: select the plant cells that have been transformed on a selective medium; regenerate the plant cells that have been transformed to produce differentiated plants; select transformed plants expressing the transgene producing the desired level of desired polypeptide(s) in the desired tissue and cellular location.

The cells that have been transformed may be grown into plants in accordance with conventional techniques. See, for example, McCormick et al. Plant Cell Reports 5:81-84 (1986). These plants may then be grown, and either pollinated with the same transformed variety or different varieties, and the resulting hybrid having constitutive expression of the desired phenotypic characteristic identified. Two or more generations may be grown to ensure that constitutive expression of the desired phenotypic characteristic is stably maintained and inherited and then seeds harvested to ensure constitutive expression of the desired phenotypic characteristic has been achieved.

In some scenarios, it may be advantageous to insert the putative bicarbonate transporter into hybrid plant lines. Hybrid lines can be created by crossing a line containing the putative bicarbonate transporter gene with a line that does not contain the putative bicarbonate transporter gene. In other cases is may be useful to cross plant lines, both of which contain a putative bicarbonate transporter gene to create hybrid plants wherein the putative bicarbonate transporter gene in each of the starting plant lines may be the same or different and may have the same promoter or a different promoter. Use of lines that possess cytoplasmic male sterility (Esser, K. et al., 2006, Progress in Botany, Springer Berlin Heidelberg. 67, 31-52) with the appropriate maintainer and restorer lines allows these hybrid lines to be produced efficiently. Cytoplasmic male sterility systems are already available for some Brassicaceae species (Esser, K. et al., 2006, Progress in Botany, Springer Berlin Heidelberg. 67, 31-52). These Brassicaceae species can be used as gene sources to produce cytoplasmic male sterility systems for other oilseeds of interest such as Camelina.

Transgenic plants can be produced using conventional techniques to express any genes of interest in plants or plant cells (Methods in Molecular Biology, 2005, vol. 286, Transgenic Plants: Methods and Protocols, Pena L., ed., Humana Press, Inc. Totowa, N.J.; Shyamkumar Barampuram and Zhanyuan J. Zhang, Recent Advances in Plant Transformation, in James A. Birchler (ed.), Plant Chromosome Engineering: Methods and Protocols, Methods in Molecular Biology, vol. 701, Springer Science+Business Media). Typically, gene transfer, or transformation, is carried out using explants capable of regeneration to produce complete, fertile plants. Generally, a DNA or an RNA molecule to be introduced into the organism is part of a transformation vector. A large number of such vector systems known in the art may be used, such as plasmids. The components of the expression system can be modified, e.g., to increase expression of the introduced nucleic acids. For example, truncated sequences, nucleotide substitutions or other modifications may be employed. Expression systems known in the art may be used to transform virtually any plant cell under suitable conditions. A transgene comprising a DNA molecule encoding a gene of interest is preferably stably transformed and integrated into the genome of the host cells. Transformed cells are preferably regenerated into whole fertile plants. Detailed description of transformation techniques are within the knowledge of those skilled in the art.

Plant promoters can be selected to control the expression of the transgene in different plant tissues or organelles for all of which methods are known to those skilled in the art (Gasser & Fraley, 1989, Science 244: 1293-1299). In one embodiment, promoters are selected from those of eukaryotic or synthetic origin that are known to yield high levels of expression in plants and algae. In a preferred embodiment, promoters are selected from those that are known to provide high levels of expression in monocots.

Chemical-regulated promoters can be used to modulate the expression of a gene in a plant through the application of an exogenous chemical regulator. Depending upon the objective, the promoter may be a chemical-inducible promoter, where application of the chemical induces gene expression, or a chemical-repressible promoter, where application of the chemical represses gene expression. Chemical-inducible promoters are known in the art and include, but are not limited to, the maize 1n2-2 promoter, which is activated by benzenesulfonamide herbicide safeners, the maize GST promoter, which is activated by hydrophobic electrophilic compounds that are used as pre-emergent herbicides, and the tobacco PR-1 promoter which is activated by salicylic acid. Other chemical-regulated promoters include steroid-responsive promoters [see, for example, the glucocorticoid-inducible promoter (Schena et al., 1991, Proc. Natl. Acad. Sci. USA 88: 10421-10425; McNellis et al., 1998, Plant J. 14:247-257) and tetracycline-inducible and tetracycline-repressible promoters (see, for example, Gatz et al., 1991, Mol. Gen. Genet. 227: 229-237; U.S. Pat. Nos. 5,814,618 and 5,789,156, herein incorporated by reference in their entirety). A three-component osmotically inducible expression system suitable for plant metabolic engineering has recently been reported (Feng et al., 2011, PLoS ONE 6: 1-9).

Constitutive promoters include, for example, the core promoter of the Rsyn7 promoter and other constitutive promoters disclosed in WO 99/43838 and U.S. Pat. No. 6,072,050, the core CaMV 35S promoter (Odell et al., 1985, Nature 313: 810-812), rice actin (McElroy et al., 1990, Plant Cell 2: 163-171), ubiquitin (Christensen et al., 1989, Plant Mol. Biol. 12: 619-632; Christensen et al., 1992, Plant Mol. Biol. 18: 675-689), pEMU (Last et al., 1991, Theor. Appl. Genet. 81: 581-588), MAS (Velten et al., 1984, EMBO J. 3: 2723-2730), and ALS promoter (U.S. Pat. No. 5,659,026). Other constitutive promoters are described in U.S. Pat. Nos. 5,608,149; 5,608,144; 5,604,121; 5,569,597; 5,466,785; 5,399,680; 5,268,463; and 5,608,142.

“Tissue-preferred” promoters can be used to target gene expression within a particular tissue. Compared to chemically inducible systems, developmentally and spatially regulated stimuli are less dependent on penetration of external factors into plant cells. Tissue-preferred promoters include those described by Van Ex et al., 2009, Plant Cell Rep. 28: 1509-1520; Yamamoto et al., 1997, Plant J. 12: 255-265; Kawamata et al., 1997, Plant Cell Physiol. 38: 792-803; Hansen et al., 1997, Mol. Gen. Genet. 254: 337-343; Russell et al., 199), Transgenic Res. 6: 157-168; Rinehart et al., 1996, Plant Physiol. 112: 1331-1341; Van Camp et al., 1996, Plant Physiol. 112: 525-535; Canevascini et al., 1996, Plant Physiol. 112: 513-524; Yamamoto et al., 1994, Plant Cell Physiol. 35: 773-778; Lam, 1994, Results Probl. Cell Differ. 20: 181-196, Orozco et al., 1993, Plant Mol. Biol. 23: 1129-1138; Matsuoka et al., 1993, Proc. Natl. Acad. Sci. USA 90: 9586-9590, and Guevara-Garcia et al., 1993, Plant J. 4: 495-505. Such promoters can be modified, if necessary, for weak expression.

Certain embodiments use transgenic plants or plant cells having multi-gene expression constructs harboring more than one transgene and promoter. The promoters can be the same or different.

Any of the described promoters can be used to control the expression of one or more of the genes of the invention, their homologs and/or orthologs as well as any other genes of interest in a defined spatiotemporal manner.

Nucleic acid sequences intended for expression in transgenic plants are first assembled in expression cassettes behind a suitable promoter active in plants. The expression cassettes may also include any further sequences required or selected for the expression of the transgene. Such sequences include, but are not restricted to, transcription terminators, extraneous sequences to enhance expression such as introns, vital sequences, and sequences intended for the targeting of the gene product to specific organelles and cell compartments. These expression cassettes can then be transferred to the plant transformation vectors described infra. The following is a description of various components of typical expression cassettes.

A variety of transcriptional terminators are available for use in expression cassettes. These are responsible for the termination of transcription beyond the transgene and the correct polyadenylation of the transcripts. Appropriate transcriptional terminators are those that are known to function in plants and include the CaMV 35S terminator, the tml terminator, the nopaline synthase terminator and the pea rbcS E9 terminator. These are used in both monocotyledonous and dicotyledonous plants.

The coding sequence of the selected gene may be genetically engineered by altering the coding sequence for optimal expression in the crop species of interest. Methods for modifying coding sequences to achieve optimal expression in a particular crop species are well known (Perlak et al., 1991, Proc. Natl. Acad. Sci. USA 88: 3324 and Koziel et al., 1993, Biotechnology 11: 194-200).

Individual plants within a population of transgenic plants that express a recombinant gene(s) may have different levels of gene expression. The variable gene expression is due to multiple factors including multiple copies of the recombinant gene, chromatin effects, and gene suppression. Accordingly, a phenotype of the transgenic plant may be measured as a percentage of individual plants within a population. The yield of a plant can be measured simply by weighing. The yield of seed from a plant can also be determined by weighing. The increase in seed weight from a plant can be due to a number of factors, an increase in the number or size of the seed pods, an increase in the number of seed or an increase in the number of seed per plant. In the laboratory or greenhouse seed yield is usually reported as the weight of seed produced per plant and in a commercial crop production setting yield is usually expressed as weight per acre or weight per hectare.

A recombinant DNA construct including a plant-expressible gene or other DNA of interest is inserted into the genome of a plant by a suitable method. Suitable methods include, for example, Agrobacterium tumefaciens-mediated DNA transfer, direct DNA transfer, liposome-mediated DNA transfer, electroporation, co-cultivation, diffusion, particle bombardment, microinjection, gene gun, calcium phosphate coprecipitation, viral vectors, and other techniques. Suitable plant transformation vectors include those derived from a Ti plasmid of Agrobacterium tumefaciens. In addition to plant transformation vectors derived from the Ti or root-inducing (Ri) plasmids of Agrobacterium, alternative methods can be used to insert DNA constructs into plant cells. A transgenic plant can be produced by selection of transformed seeds or by selection of transformed plant cells and subsequent regeneration.

In one embodiment, the transgenic plants are grown (e.g., on soil) and harvested. In one embodiment, above ground tissue is harvested separately from below ground tissue. Suitable above ground tissues include shoots, stems, leaves, flowers, grain, and seed. Exemplary below ground tissues include roots and root hairs. In one embodiment, whole plants are harvested and the above ground tissue is subsequently separated from the below ground tissue.

Genetic constructs may encode a selectable marker to enable selection of transformation events. There are many methods that have been described for the selection of transformed plants [for review see (Miki et al., Journal of Biotechnology, 2004, 107, 193-232) and references incorporated within]. Selectable marker genes that have been used extensively in plants include the neomycin phosphotransferase gene nptll (U.S. Pat. Nos. 5,034,322, 5,530,196), hygromycin resistance gene (U.S. Pat. No. 5,668,298, Waldron et al., (1985), Plant Mol Biol, 5:103-108; Zhijian et al., (1995), Plant Sci, 108:219-227), the bar gene encoding resistance to phosphinothricin (U.S. Pat. No. 5,276,268), the expression of aminoglycoside 3″-adenyltransferase (aadA) to confer spectinomycin resistance (U.S. Pat. No. 5,073,675), the use of inhibition resistant 5-enolpyruvyl-3-phosphoshikimate synthetase (U.S. Pat. No. 4,535,060) and methods for producing glyphosate tolerant plants (U.S. Pat. Nos. 5,463,175; 7,045,684). Other suitable selectable markers include, but are not limited to, genes encoding resistance to chloramphenicol (Herrera Estrella et al., (1983), EMBO J, 2:987-992), methotrexate (Herrera Estrella et al., (1983), Nature, 303:209-213; Meijer et al, (1991), Plant Mol Biol, 16:807-820); streptomycin (Jones et al., (1987), Mol Gen Genet, 210:86-91); bleomycin (Hille et al., (1990), Plant Mol Biol, 7:171-176); sulfonamide (Guerineau et al., (1990), Plant Mol Biol, 15:127-136); bromoxynil (Stalker et al., (1988), Science, 242:419-423); glyphosate (Shaw et al., (1986), Science, 233:478-481); phosphinothricin (DeBlock et al., (1987), EMBO J, 6:2513-2518).

Methods of plant selection that do not use antibiotics or herbicides as a selective agent have been previously described and include expression of glucosamine-6-phosphate deaminase to inactive glucosamine in plant selection medium (U.S. Pat. No. 6,444,878) and a positive/negative system that utilizes D-amino acids (Erikson et al., Nat Biotechnol, 2004, 22, 455-8). European Patent Publication No. EP 0 530 129 A1 describes a positive selection system which enables the transformed plants to outgrow the non-transformed lines by expressing a transgene encoding an enzyme that activates an inactive compound added to the growth media. U.S. Pat. No. 5,767,378 describes the use of mannose or xylose for the positive selection of transgenic plants.

Methods for positive selection using sorbitol dehydrogenase to convert sorbitol to fructose for plant growth have also been described (WO 2010/102293). Screenable marker genes include the beta-glucuronidase gene (Jefferson et al., 1987, EMBO J. 6: 3901-3907; U.S. Pat. No. 5,268,463) and native or modified green fluorescent protein gene (Cubitt et al., 1995, Trends Biochem. Sci. 20: 448-455; Pan et al., 1996, Plant Physiol. 112: 893-900).

Transformation events can also be selected through visualization of fluorescent proteins such as the fluorescent proteins from the nonbioluminescent Anthozoa species which include DsRed, a red fluorescent protein from the Discosoma genus of coral (Matz et al. (1999), Nat Biotechnol 17: 969-73). An improved version of the DsRed protein has been developed (Bevis and Glick (2002), Nat Biotech 20: 83-87) for reducing aggregation of the protein.

Visual selection can also be performed with the yellow fluorescent proteins (YFP) including the variant with accelerated maturation of the signal (Nagai, T. et al. (2002), Nat Biotech 20: 87-90), the blue fluorescent protein, the cyan fluorescent protein, and the green fluorescent protein (Sheen et al. (1995), Plant J 8: 777-84; Davis and Vierstra (1998), Plant Molecular Biology 36: 521-528). A summary of fluorescent proteins can be found in Tzfira et al. (Tzfira et al. (2005), Plant Molecular Biology 57: 503-516) and Verkhusha and Lukyanov (Verkhusha, V. V. and K. A. Lukyanov (2004), Nat Biotech 22: 289-296) whose references are incorporated in entirety. Improved versions of many of the fluorescent proteins have been made for various applications. Use of the improved versions of these proteins or the use of combinations of these proteins for selection of transformants will be obvious to those skilled in the art.

The plants modified for enhanced yield may have stacked input traits that include herbicide resistance and insect tolerance, for example a plant that is tolerant to the herbicide glyphosate and that produces the Bacillus thuringiensis (BT) toxin. Glyphosate is a herbicide that prevents the production of aromatic amino acids in plants by inhibiting the enzyme 5-enolpyruvylshikimate-3-phosphate synthase (EPSP synthase). The overexpression of EPSP synthase in a crop of interest allows the application of glyphosate as a weed killer without killing the modified plant (Suh, et al., J. M Plant Mol. Biol. 1993, 22, 195-205). BT toxin is a protein that is lethal to many insects providing the plant that produces it protection against pests (Barton, et al. Plant Physiol. 1987, 85, 1103-1109). Other useful herbicide tolerance traits include but are not limited to tolerance to Dicamba by expression of the dicamba monoxygenase gene (Behrens et al, 2007, Science, 316, 1185), tolerance to 2,4-D and 2,4-D choline by expression of a bacterial aad-1 gene that encodes for an aryloxyalkanoate dioxygenase enzyme (Wright et al., Proceedings of the National Academy of Sciences, 2010, 107, 20240), glufosinate tolerance by expression of the bialophos resistance gene (bar) or the pat gene encoding the enzyme phosphinotricin acetyl transferase (Droge et al., Planta, 1992, 187, 142), as well as genes encoding a modified 4-hydroxyphenylpyruvate dioxygenase (HPPD) that provides tolerance to the herbicides mesotrione, isoxaflutole, and tembotrione. (Siehl et al., Plant Physiol, 2014, 166, 1162).

The transgenic land plant that comprises a putative bicarbonate transporter protein of an edible eukaryotic algae, as disclosed, can be modified for further enhanced yield.

One approach for further enhanced yield comprises modifying the transgenic land plant for reduced expression of cell wall invertase inhibitor (also termed cwii). It is believed that expression of a novel class of cell wall invertase inhibitors is upregulated in plants modified to express CCP1 of Chlamydomonas reinhardtii and/or putative bicarbonate transporter proteins of edible eukaryotic algae that are orthologs of CCP1, and that downregulating cell wall invertase inhibitor genes in plants modified to express CCP1 of Chlamydomonas reinhardtii and/or putative bicarbonate transporter proteins of edible eukaryotic algae that are orthologs of CCP1 would result in further enhanced yield, as discussed below.

Cell wall invertase inhibitors of plants such as tomato and rice are known in the art, as taught for example by Wang et al. (2008), Nature Genetics 40(11):1370-1374, and Jin et al. (2009), Plant Cell 21(7):2072-2089, and can be identified in other plants, for example based on homology, in accordance with methods known in the art.

Modifying the transgenic land plant for reduced expression of cell wall invertase inhibitor can be accomplished, for example, by expressing a suppressor of an endogenous cell wall invertase inhibitor of the transgenic land plant, for example by antisense RNA or RNA interference, in accordance with methods known in the art. Such modification also can be accomplished, for example, by expressing a modified cell wall invertase inhibitor in place of an endogenous cell wall invertase inhibitor of the transgenic land plant, for example by CRISPR-associated protein 9 modification of a gene encoding the endogenous cell wall invertase inhibitor, also in accordance with methods known in the art.

Accordingly, in some examples the transgenic land plant is modified to express (i) a suppressor of an endogenous cell wall invertase inhibitor of the transgenic land plant or (ii) a modified cell wall invertase inhibitor in place of an endogenous cell wall invertase inhibitor of the transgenic land plant. In some of these examples relating to a suppressor of the endogenous cell wall invertase inhibitor, the suppressor is (i) an antisense RNA complementary to messenger RNA of the endogenous cell wall invertase inhibitor or (ii) an RNA interference nucleic acid that reduces expression of messenger RNA of the endogenous cell wall invertase inhibitor. Also, in some of these examples relating to a modified cell wall invertase inhibitor, the modified cell wall invertase inhibitor has been modified by transforming the transgenic land plant with a nucleotide sequence encoding CRISPR-associated protein 9 under the control of a promoter and with a nucleotide sequence encoding a single guide RNA under the control of a promoter, wherein the single guide RNA comprises 19 to 22 nucleotides and is fully homologous to a region of a gene encoding the endogenous cell wall invertase inhibitor.

Another approach for further enhanced yield comprises modifying the transgenic land plant to express carbonic anhydrase targeted to mitochondria. As noted above, the carbon-concentrating mechanism of eukaryotic algae includes expression of a and carbonic anhydrases for concentration of bicarbonate in chloroplast stroma. More specifically, carbonic anhydrases catalyze reversible hydration of CO₂ to bicarbonate and play a central role in controlling pH balance and inorganic carbon sequestration and flux. It is believed that expressing carbonic anhydrase targeted to mitochondria in plants modified to express CCP1 of Chlamydomonas reinhardtii and/or putative bicarbonate transporter proteins of edible eukaryotic algae that are orthologs of CCP1 would further enhance availability of bicarbonate (and/or other small molecules) for CCP1 and/or the putative bicarbonate transporter proteins of edible eukaryotic algae that are orthologs of CCP1 to export to cytosol of cells.

Carbonic anhydrase of plants such as rice, maize, soybean, tomato, barley, cucumber, alfalfa, bean, pea, pear, almond, mung bean, tobacco, cotton, aspen, and Arabidopsis are known in the art, as taught for example by Schroeder, U.S. Pat. No. 8,916,745 and references cited therein, and can be identified in other plants, for example based on homology, in accordance with methods known in the art.

Modifying the transgenic land plant to express carbonic anhydrase targeted to mitochondria can be carried out by methods that are known in the art, as discussed above. The carbonic anhydrase can be, for example, a carbonic anhydrase that is targeted to mitochondria based on including an endogenous mitochondrial targeting signal, or a carbonic anhydrase that is targeted to mitochondria based on having been engineered to include a mitochondrial targeting signal. The carbonic anhydrase also can be, for example, a plant carbonic anhydrase. The plant carbonic anhydrase can be, for example, a carbonic anhydrase of an edible plant, such as rice, maize, soybean, tomato, barley, cucumber, alfalfa, bean, pea, pear, almond, or mung bean, or a carbonic anhydrase of another plant, such as tobacco, cotton, aspen, or Arabidopsis. Consistent with the transgenic land plant, the carbonic anhydrase can be, for example, a carbonic anhydrase of an edible eukaryotic algae.

Accordingly, in some examples the transgenic land plant is modified to express carbonic anhydrase targeted to mitochondria. Also in some examples, the carbonic anhydrase is a carbonic anhydrase of rice, maize, soybean, canola, camelina, tomato, barley, cucumber, alfalfa, bean, pea, pear, almond, or mung bean that is targeted to mitochondria. Also in some examples, the carbonic anhydrase is a carbonic anhydrase of tobacco, cotton, aspen, or Arabidopsis that is targeted to mitochondria. Also in some examples, the carbonic anhydrase is a carbonic anhydrase of an edible eukaryotic algae that is targeted to mitochondria.

### EXAMPLES

### Example 1. CCP1 Orthologs in Edible Algae

### Edible Eukaryotic Algae Homology Searches

Various BLAST searches (e.g. tblastn; website://blast.ncbi.nlm.nih.gov/Blast.cgi) were conducted using a translated nucleotide database, a whole genome shotgun (also termed WGS) database, and a transcriptome assembly (also termed TSA) database to find sequences highly similar to the CCP1 protein from Chlamydomonas reinhardtii in edible algae species (Table 1). The percent homology of the translated edible algae sequence was compared to the CCP1 amino acid sequence using the alignment feature of VectorNTI software. Sequences containing between 60% and 18% homology were obtained, as shown in TABLE 1. Several publically available web programs were used to predict putative transmembrane regions to further characterize the edible algae sequences including Motif Finder (website://www.genome.jp/tools/motif/; TABLE 1), ProSite (website://prosite.expasy.org/; TABLE 1), and Phobius (website://phobius.sbc.su.se/; FIG. 1, FIG. 2, FIG. 3, FIG. 4, and FIG. 5). The Motif Finder program predicts both CCP1 and the edible algae orthologs as Milo_carr (PF00153) or mitochondrial carrier proteins (TABLE 1). This class of proteins carries molecules across the membrane of mitochondria (website://pfam.xfam.org/family/PF00153). The ProSite program predicted both CCP1 and the edible algae orthologs as SOLCAR (PS50920) or solute carrier proteins (TABLE 1). This class of proteins are defined as substrate carrier proteins involved in energy transfer in the inner mitochondrial membrane website://prosite.expasy.org/cgi-bin/prosite/nicedoc.pl?PS50920). Mapping of predicted transmembrane regions of CCP1 and comparing the results to the orthologs with the highest homology was used to further characterize the proteins (FIG. 1-5). Based on the combined analyses of TABLE 1 and FIG. 1, FIG. 2, FIG. 3, FIG. 4, and FIG. 5, the Chlorella sorokiniana protein encoded by gene GAPD01006726.1 is the most similar to the Chlamydomonas reinhardtii protein encoded by gene XM_001692145.1.

### Multiple Sequence Alignment

A multiple sequence alignment of CCP1 of Chlamydomonas reinhardtii and eight orthologs of CCP1 of eukaryotic edible algae as identified based on homology searches was prepared using a Multiple Sequence Alignment tool (EMBL-EBI; website://www.ebi.ac.uk/Tools/msa/clustalo/). Specifically, CCP1 of Chlamydomonas reinhardtii of SEQ ID NO: 1, a putative bicarbonate transporter protein of an edible Chlorella sorokiniana of SEQ ID NO: 2, putative bicarbonate transporter proteins of an edible Chlorella variabilis of SEQ ID NO: 3, SEQ ID NO: 4, SEQ ID NO: 5, and SEQ ID NO: 6, and putative bicarbonate transporter proteins of an edible Chondrus crispus of SEQ ID NO: 7, SEQ ID NO: 8, and SEQ ID NO: 9, were aligned by CLUSTAL, using default parameters (dealign input sequences [no]; MBED-like clustering guide-tree [yes]; MBED-like clustering iteration [yes]; number of combined iterations [default(0)]; max guide tree iterations [default (−1)]; max HMM iterations [default(−1)]; and order [aligned]). Results are shown in FIGS. 6A-B.

With reference to FIGS. 6A-B and TABLE 1, structural features and characteristics shared among the various orthologs of CCP1 include (i) (a) a proline residue at position 268, (b) an aspartate residue or glutamine residue at position 270, (c) a lysine residue or arginine residue at position 273, and (d) a serine residue or threonine residue at position 274, with numbering of positions relative to CCP1 of Chlamydomonas reinhardtii of SEQ ID NO: 1, and (ii) an overall identity of at least 15%. With reference to FIG. 1, FIG. 2, FIG. 3, FIG. 4, and FIG. 5 and TABLE 1, structural features and characteristics shared among the various orthologs of CCP1 also include a potential transmembrane region between about positions 245 to 265, with numbering of positions relative to CCP1 of Chlamydomonas reinhardtii of SEQ ID NO: 1. The noted amino acid residues, i.e. proline residue at position 268, aspartate residue or glutamine residue at position 270, lysine residue or arginine residue at position 273, and serine residue or threonine residue at position 274, with numbering of positions relative to CCP1 of Chlamydomonas reinhardtii of SEQ ID NO: 1, occur at or after the C-terminal portion of this potential transmembrane region of each of CCP1 and the orthologs. Conservation of the noted amino acid residues, in combination with an overall identity of at least 15%, suggests a structure/function relationship shared among CCP1 and the orthologs.

TABLE 1
Proteins with homology to Chlamydomonas reinhardtii CCP1 in edible algae

| | Nucleotide | | | | | | | |
|---|---|---|---|---|---|---|---|---|
| | Accession | | Homology to CCP1 | | | | | |
| | (and SEQ ID NO of | Number | Consensus | Identity | | | | |
| | corresponding | of amino | positions | Positions | Program | | | |
| Organism | protein) | acids | (%) | (%) | Motif Finder^{b} | | ProSite^{c} | |
| Chlamydomonas | XM_001692145.1 | 358 | 100 | 100 | Mitochondrial | 3 predicted | Solute | 3 predicted motifs |
| reinhardtii | (SEQ ID NO: 1) | | | | carrier protein | motifs spanning | carrier | spanning amino |
| | | | | | | amino acids 28- | protein^{d} | acids 22-118; |
| | | | | | | 119; 129-235; & | | 131-231; & 246-333 |
| | | | | | | 245-334 | | |
| Chlorella | GAPD01006726.1 | 354^{a} | 72.8 | 59.9 | Mitochondrial | 3 predicted | Solute | 3 predicted motifs |
| sorokiniana | (SEQ ID NO: 2) | | | | carrier protein | motifs spanning | carrier | spanning amino |
| | | | | | | amino acids 25- | protein^{d} | acids 20-116; |
| | | | | | | 117; 128-228; & | | 131-227; & 238-325 |
| | | | | | | 243-329 | | |
| Chlorella | XM_005846489.1 | 303 | 42.5 | 25.8 | Mitochondrial | 3 predicted | Solute | 3 predicted motifs |
| variabilis | (SEQ ID NO: 6) | | | | carrier protein | motifs spanning | carrier | spanning amino |
| | | | | | | amino acids 4- | protein^{d} | acids 3-86; 96- |
| | | | | | | 88; 97-199; & | | 200; & 212-300 |
| | | | | | | 212-301 | | |
| Chlorella | XM_005852157.1 | 323 | 40.3 | 25.2 | Mitochondrial | 3 predicted | Solute | 3 predicted motifs |
| variabilis | (SEQ ID NO: 4) | | | | carrier protein | motifs spanning | carrier | spanning amino |
| | | | | | | amino acids 26- | protein^{d} | acids 25-112; |
| | | | | | | 115; 125-221; & | | 124-218; & 230-319 |
| | | | | | | 229-322 | | |
| Chlorella | XM_005843001.1 | 323 | 39.3 | 24.7 | Mitochondrial | 3 predicted | Solute | 3 predicted motifs |
| variabilis | (SEQ ID NO: 5) | | | | carrier protein | motifs spanning | carrier | spanning amino |
| | | | | | | amino acids 9- | protein^{d} | acids 8-92; 101- |
| | | | | | | 90; 108-187; & | | 189; & 221-308 |
| | | | | | | 225-307 | | |
| Chondrus | XM_005712871.1 | 328 | 34.7 | 20.3 | Mitochondrial | 3 predicted | Solute | 3 predicted motifs |
| crispus | (SEQ ID NO: 7) | | | | carrier protein | motifs spanning | carrier | spanning amino |
| | | | | | | amino acids 40- | protein^{d} | acids 39-128; 135- |
| | | | | | | 127; 137-230; & | | 227; & 238-325 |
| | | | | | | 239-326 | | |
| Chlorella | XM_005851446.1 | 306 | 35.8 | 21.7 | Mitochondrial | 3 predicted | Solute | 3 predicted motifs |
| variabilis | (SEQ ID NO: 3) | | | | carrier protein | motifs spanning | carrier | spanning amino |
| | | | | | | amino acids 11- | protein^{d} | acids 11-100; |
| | | | | | | 101; 112-206; & | | 112-203; & 212- |
| | | | | | | 213-299 | | 298 |
| Chondrus | XM_005715654.1 | 233 | 35.2 | 22.9 | Mitochondrial | 3 predicted | Solute | 3 predicted motifs |
| crispus | (SEQ ID NO: 8) | | | | carrier protein | motifs spanning | carrier | spanning amino |
| | | | | | | amino acids 3- | protein^{d} | acids 1-37; 47- |
| | | | | | | 40; 47-132; & | | 131; & 142-229 |
| | | | | | | 141-231 | | |
| Chondrus | XM_005713259.1 | 194 | 29.9 | 18.4 | Mitochondrial | 2 predicted | Solute | 2 predicted motifs |
| crispus | (SEQ ID NO: 9) | | | | carrier protein | motifs spanning | carrier | spanning amino |
| | | | | | | amino acids 7-93 | protein^{d} | acids 8-92 & 103- |
| | | | | | | & 102-191 | | 190 |

^{a}sequence from first methionine of deposited transcribed mRNA sequence to first stop codon
^{b}website://www.genome.jp/tools/motif/
^{c}website://prosite.expasy.org/
^{d}predicted as one of several substrate carrier proteins involved in energy transfer in the inner mitochondrial membrane (website://prosite.expasy.org/cgi-bin/prosite/nicedoc.pl?PS50920)

### Example 2. Preparation of Genetic Constructs Encoding Edible Algae Orthologs of CCP1

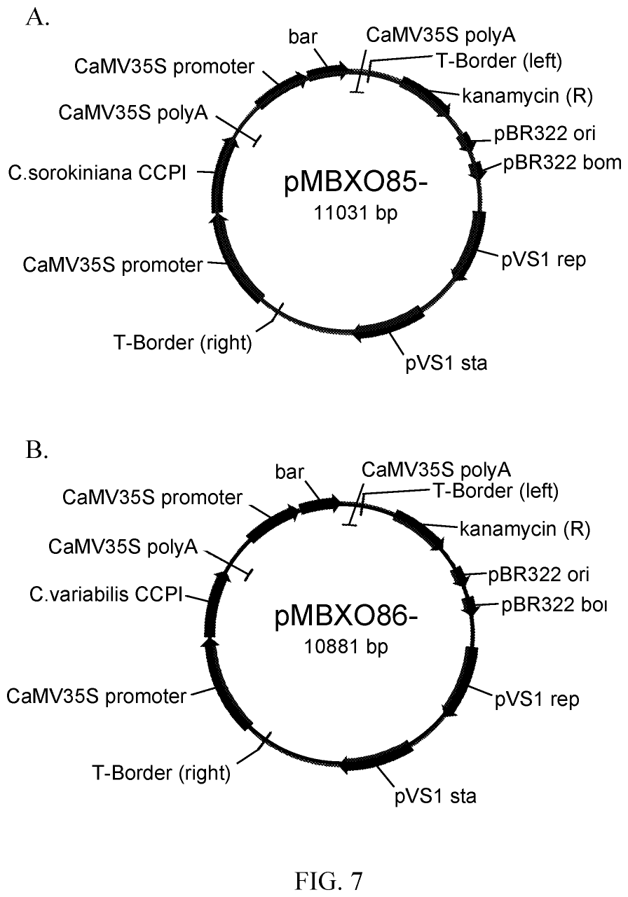

Genetic constructs pMBXO85 and pMBXO86 contain orthologs of CCP1 from edible algae and are derivatives of pCAMBIA binary vectors (Centre for Application of Molecular Biology to International Agriculture, Canberra, Australia). These plasmids were constructed using cloning techniques that are standard to those skilled in the art. The source of orthologs of the CCP1 gene encoded by these genetic constructs, as well as the promoter driving the expression of the CCP1 ortholog, are listed in TABLE 2. Both pMBXO85 and pMBXO86 have a constitutive expression cassette for the bar gene, that imparts transgenic plants resistance to the herbicide bialophos allowing for their selection. Maps of pMBXO85 and pMBXO86 illustrating the plant expression elements for directing the expression of the CCP1 orthologs in plants are shown in FIG. 7. Nucleotide sequences of pMBXO85 (SEQ ID NO: 10) and pMBXO86 (SEQ ID NO: 11) are also provided.

TABLE 2
Summary of constructs for transformation into Camelina

| | | | Nucleotide Accession |
|---|---|---|---|
| Construct | | Source of CCP1 | (and SEQ ID NO of |
| name | Promoter | ortholog gene | corresponding protein) |
| pMBXO85 | 35sCAMV | Chlorella | GAPD01006726.1 |
| | (constitutive) | sorokiniana | (SEQ ID NO: 2) |
| pMBXO86 | 35sCAMV | Chlorella variabilis | XM_005846489.1 |
| | (constitutive) | | (SEQ ID NO: 6) |

### Example 3. Transformation of Genetic Constructs Encoding Edible Algae Orthologs of CCP1 into Camelina sativa

In preparation for plant transformation experiments, seeds of Camelina sativa germplasm 10CS0043 (abbreviated WT43, obtained from Agriculture and Agri-Food Canada) were sown directly into 4 inch (10 cm) pots filled with soil in the greenhouse. Growth conditions were maintained at 24° C. during the day and 18° C. during the night. Plants were grown until flowering. Plants with a number of unopened flower buds were used in ‘floral dip’ transformations.

Agrobacterium strain GV3101 (pMP90) was transformed with either pMBXO85 or pMBXO86 using electroporation. A single colony of GV3101 (pMP90) containing the construct of interest was obtained from a freshly streaked plate and was inoculated into 5 mL LB medium. After overnight growth at 28° C., 2 mL of culture was transferred to a 500-mL flask containing 300 mL of LB and incubated overnight at 28° C. Cells were pelleted by centrifugation (6,000 rpm, 20 min), and diluted to an OD600 of ˜0.8 with infiltration medium containing 5% sucrose and 0.05% (v/v) Silwet-L77 (Lehle Seeds, Round Rock, Tex., USA). Camelina plants were transformed by “floral dip” using the pMBXO85 and pMBXO86 transformation constructs as follows. Pots containing plants at the flowering stage were placed inside a 460 mm height vacuum desiccator (Bel-Art, Pequannock, N.J., USA). Inflorescences were immersed into the Agrobacterium inoculum contained in a 500-ml beaker. A vacuum (85 kPa) was applied and held for 5 min. Plants were removed from the desiccator and were covered with plastic bags in the dark for 24 h at room temperature. Plants were removed from the bags and returned to normal growth conditions within the greenhouse for seed formation (T1 generation of seed).

T1 seeds were planted in soil and transgenic plants were selected by spraying a solution of 400 mg/L of the herbicide Liberty (active ingredient 15% glufosinate-ammonium). This allows identification of transgenic plants containing the bar gene on the T-DNA in the plasmid vectors pMBXO85 and pMBXO86 (FIG. 7). Transgenic plant lines were further confirmed using PCR with primers specific to the edible algae ortholog gene of interest. PCR positive lines were grown in a greenhouse to produce the next generation of seed (T2 seed). Seeds were isolated from each plant and were dried in an oven with mechanical convection set at 22° C. for two days. The weight of the entire harvested seed obtained from individual plants was measured and recorded. Multiple T1 plants from pMBXO85 and pMBXO86 plants produced more T2 seed than wild-type controls. The best line from the pMBXO85 transformation produced 54% more seed than wild-type controls whereas the best pMBXO86 line produced 30% more seed than controls.

TABLE 3
T2 seed yield in lines of Camelina transformed
with pMBXO85 and pMBXO86.

| Genetic | | Seed | % of wild-type |
|---|---|---|---|
| Construct | Line | Yield (g) | control |
| None | Wild-type¹ | 4.39 ± 1.42 | 100 |
| pMBXO85 | 16-0889 | 6.76 | 154 |
| | 16-0886 | 6.15 | 140 |
| | 16-0894 | 6.1 | 139 |
| | 16-0895 | 5.82 | 133 |
| | 16-0896 | 5.48 | 125 |
| | 16-0888 | 5.24 | 119 |
| | 16-0891 | 5.24 | 119 |
| | 16-0897 | 5.1 | 116 |
| | 16-0903 | 4.86 | 111 |
| | 16-0893 | 4.81 | 110 |
| | 16-0932 | 4.76 | 109 |
| | 16-0892 | 4.61 | 105 |
| | 16-0920 | 4.55 | 104 |
| pMBXO86 | 16-0839 | 5.68 | 130 |
| | 16-0853 | 4.7 | 107 |

¹Wild-type control seed yield values are an average of 25 plants.

### Example 4. Methods for Characterizing CCP1 Transport

Some mitochondrial and plastid carrier proteins have previously been shown to functionally localize into the E. coli cytoplasmic membrane including mitochondrial ADP/ATP carriers (Haferkamp et al. (2002), European Journal of Biochemistry 269, 3172; Razakantoanina, et al. (2008), Experimental Parasitology 118, 181), plastid ATP/ADP transporter genes (Tjaden, et al. (1998), J Biol Chem 273, 9630), and some bicarbonate transporters (Du et al. (2014), PLoS One 9, e115905).

Cyanobacterial bicarbonate transporters have been characterized in Escherichia coli using a mutant E. coli strain, termed EDCM636, that is deficient in carbonic anhydrase activity (Du, J. et al. (2014)). This mutant is unable to grow on LB or M9 plates without supplementation with high levels of CO₂. As reported by Du et al. (2014), expression of six cyanobacterial bicarbonate transporters, corresponding to β forms of SbtA of Synechococcus sp. WH5701, Cyanobium sp. PCC 7001, Cyanobium sp. PCC 6307, Synechococcus elongatus PCC 7942, Synechocystis sp. PCC 6803, and Synechococcus sp. PCC 7002, restored growth of the E. coli mutant at atmospheric levels of CO₂, whereas expression of various others did not.

The function of CCP1 and potential orthologs thereof with respect to bicarbonate or other small molecule transport may be tested by an analogous approach, and corresponding functional screens developed, also based on restoring growth of a mutant E. coli strain that is deficient in an enzymatic activity that prevents that production of a small molecule required for growth. For example, the CCP1 coding sequence from Chlamydomonas reinhardtii can be synthesized with a sequence that is codon optimized for expression in E. coli and cloned into an E. coli expression vector. Codon optimized sequences of potential orthologs thereof can also can be synthesized and cloned into E. coli expression vectors.

For testing bicarbonate transport, codon optimized sequences of two SbtA bicarbonate transporters from Cyanobium sp. PCC 7001 (also termed SbtA_{cyanobium sp.PCC 7001}) and Synechocystis sp. PCC 6803 (also termed SbtA_{Synechocystis sp.PCC 6803}) can be synthesized and cloned into E. coli expression vectors. These two SbtA proteins can serve as positive controls for functional heterologous expression in E. coli, based on SbtA of Cyanobium sp. PCC 7001 having a Kₘ calculated to be 189 μM and SbtA of Synechocystis sp. PCC 6803 having a Kₘ under 100 μM, and based on both previously having been shown to enable E. coli bicarbonate uptake, as taught by Du et al. The E. coli expression vector lacking a cloned sequence can serve as a negative control. Restoration of growth of the mutant E. coli strain by the CCP1 coding sequence and by potential orthologs thereof would indicate that these sequences also enable E. coli bicarbonate uptake.

Likewise, E. coli mutants deficient in the transport and/or production of small molecules, such as for example C4-dicarboxylic acids, can be used to test the ability of CCP1 to transport α-ketoglutarate, succinate, malate, and oxaloacetate. The ychM gene of E. coli has been shown to be the main succinate transporter under acidic pH growth conditions (Karinou et al., 2013, Molecular Microbiology, 87, 623) and an E. coli strain with a mutated ychM gene can be used to characterize the ability of CCP1 to transport this molecule.

Function of CCP1 and potential orthologs thereof with respect to bicarbonate transport also may be tested, and corresponding functional screens developed, based on use of yeast modified to express CCP1 and potential orthologs thereof. For example, a functional screen for CCP1 expression in yeast based on sensitivity of growth to bicarbonate works as follows. CCP1 can be expressed in yeast to examine if CCP1 utilizes HCO₃⁻ as a substrate. HCO₃⁻ is the major pH regulator of the yeast cytosol. Accordingly, disruptions in regulation of HCO₃⁻ at the mitochondrial membrane result in a loss of respiration and an inhibition of growth. Increasing concentrations of HCO₃⁻ in media should result in rapid inhibition of yeast growth in cultures expressing CCP1 relative to yeast transformed with an empty vector control. Non-specific compounds, such as borate, NaCl and nitrate, also can be used as negative controls, as these would not be expected to inhibit growth. It is expected that this screen. In accordance with this approach, function of CCP1 and/or bicarbonate transporter proteins of edible eukaryotic algae that are orthologs of CCP1 as bicarbonate transporters can be confirmed. Moreover, additional bicarbonate transporter proteins that are localized to mitochondria and that function similarly can be identified.

### Example 5. Model for Further Enhanced Yield of Plants Based on Inhibiting Expression of CWII that would Otherwise be Upregulated in CCP1 Lines

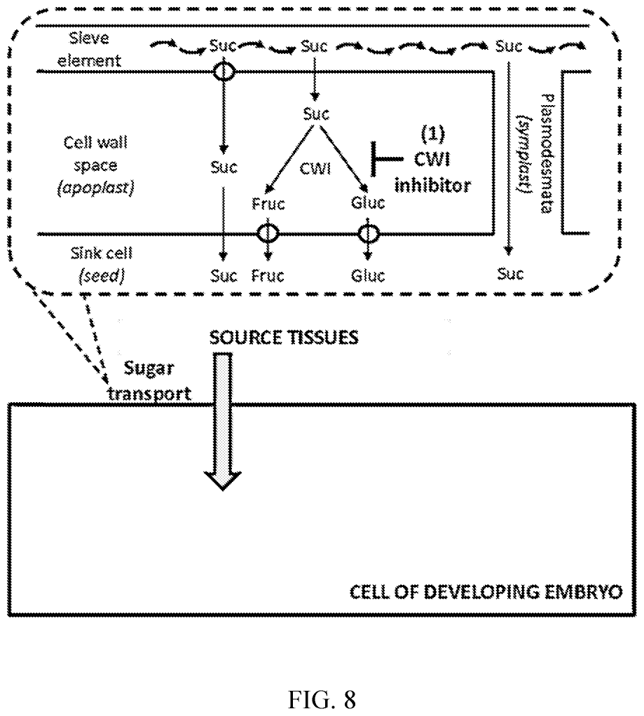

A model for further enhanced yield based on inhibiting expression of cell wall invertase inhibitor that would otherwise be upregulated in CCP1 lines is provided, with reference to FIG. 8, as follows.

It is believed that expression of a novel class of cell wall invertase inhibitors is upregulated in plants modified to express CCP1 of Chlamydomonas reinhardtii and/or putative bicarbonate transporter proteins of edible eukaryotic algae that are orthologs of CCP1. In accordance with this model, sucrose transport and allocation is a key determinant of seed yield. Export and import of sucrose through the apoplasm are controlled by cell wall invertases (also termed CWI), which hydrolyze sucrose to fructose and glucose. Activity of cell wall invertase is controlled by a cell wall invertase inhibitor. The novel class of cell wall invertase inhibitors is upregulated in plants modified to express CCP1 of Chlamydomonas reinhardtii. This is likely a response of cells to increased carbon capture. Also, cell wall invertase inhibitors are good targets for genome editing. Accordingly, it is believed that downregulating cell wall invertase inhibitor genes in plants modified to express CCP1 of Chlamydomonas reinhardtii and/or putative bicarbonate transporter proteins of edible eukaryotic algae that are orthologs of CCP1 would result in further enhanced yield.

The invention has been described with reference to the example embodiments described above. Modifications and alterations will occur to others upon a reading and understanding of this specification. Examples embodiments incorporating one or more aspects of the invention are intended to include all such modifications and alterations insofar as they come within the scope of the appended claims.

### REFERENCE TO A “SEQUENCE LISTING,” A TABLE, OR A COMPUTER PROGRAM LISTING APPENDIX SUBMITTED AS AN ASCII TEXT FILE

The material in the ASCII text file, named “YTEN-55463US1-Sequences_ST25.txt”, created Jul. 9, 2019, file size of 53,248 bytes, is hereby incorporated by reference.

1. A transgenic land plant comprising a putative transporter protein of an edible eukaryotic algae, wherein:
the putative transporter protein of the edible eukaryotic algae is heterologous with respect to the transgenic land plant;
the putative transporter protein is an ortholog of CCP1 of Chlamydomonas reinhardtii of SEQ ID NO: 1; and
the putative transporter protein is localized to mitochondria of the transgenic land plant based on a mitochondrial targeting signal intrinsic to the putative transporter protein.

2. The transgenic land plant of claim 1, wherein the putative transporter protein corresponds to a putative transporter protein selected from the group consisting of (a) a putative transporter protein of an edible Chlorella sorokiniana of SEQ ID NO: 2, (b) a putative transporter protein of an edible Chlorella variabilis of SEQ ID NO: 3, SEQ ID NO: 4, SEQ ID NO: 5, or SEQ ID NO: 6, and (c) a putative transporter protein of an edible Chondrus crispus of SEQ ID NO: 7, SEQ ID NO: 8, or SEQ ID NO: 9.

3. The transgenic land plant of claim 1, wherein the putative transporter protein is an ortholog of CCP1 of Chlamydomonas reinhardtii of SEQ ID NO: 1 based on comprising: (i) (a) a proline residue at position 268, (b) an aspartate residue or glutamine residue at position 270, (c) a lysine residue or arginine residue at position 273, and (d) a serine residue or threonine residue at position 274, with numbering of positions relative to CCP1 of Chlamydomonas reinhardtii of SEQ ID NO: 1, and (ii) an overall identity of at least 15%.

4. The transgenic land plant of claim 1, wherein the putative transporter protein is localized to mitochondria of the transgenic land plant to a greater extent than to chloroplasts of the transgenic land plant by a factor of at least 2, at least 5, or at least 10.

5. The transgenic land plant of claim 1, wherein the putative transporter protein consists essentially of an amino acid sequence that is identical to that of a wild-type edible eukaryotic algal putative transporter protein.

6. The transgenic land plant of claim 1, further comprising a heterologous polynucleotide, wherein the putative transporter protein is encoded by the heterologous polynucleotide.

7. The transgenic land plant of claim 6, wherein the heterologous polynucleotide comprises a heterologous promoter.

8. The transgenic land plant of claim 7, wherein the heterologous promoter is a chemical-regulated promoter, a constitutive promoter, or a tissue-preferred promoter.

9. The transgenic land plant of claim 6, wherein the heterologous polynucleotide is integrated into genomic DNA of the transgenic land plant.

10. The transgenic land plant of claim 1, wherein the transgenic land plant has a CO₂ assimilation rate that is at least 5% higher, at least 10% higher, at least 20% higher, or at least 40% higher, than for a corresponding reference land plant not comprising the putative transporter protein.

11. The transgenic land plant of claim 1, wherein the transgenic land plant has a transpiration rate that is at least 5% lower, at least 10% lower, at least 20% lower, or at least 40% lower, than for a corresponding reference land plant not comprising the putative transporter protein.

12. The transgenic land plant of claim 1, wherein the transgenic land plant has a seed yield that is at least 5% higher, at least 10% higher, at least 20% higher, at least 40% higher, at least 60% higher, or at least 80% higher, than for a corresponding reference land plant not comprising the putative transporter protein.

13. The transgenic land plant of claim 1, wherein the transgenic land plant is modified to express (i) a suppressor of an endogenous invertase inhibitor of the transgenic land plant or (ii) a modified invertase inhibitor in place of an endogenous invertase inhibitor of the transgenic land plant.

14. The transgenic land plant of claim 13, wherein the suppressor of the endogenous invertase inhibitor is (i) an antisense RNA complementary to messenger RNA of the endogenous invertase inhibitor or (ii) an RNA interference nucleic acid that reduces expression of messenger RNA of the endogenous invertase inhibitor.

15. The transgenic land plant of claim 13, wherein the modified invertase inhibitor has been modified by transforming the transgenic land plant with a nucleotide sequence encoding CRISPR-associated protein 9 under the control of a promoter and with a nucleotide sequence encoding a single guide RNA under the control of a promoter, wherein the single guide RNA comprises 19 to 22 nucleotides and is fully homologous to a region of a gene encoding the endogenous invertase inhibitor.

16-19. (canceled)

20. The transgenic land plant of claim 1, wherein the only heterologous algal protein that the transgenic land plant comprises is the putative transporter protein.

21. The transgenic land plant of claim 1, wherein the transgenic land plant is a C3 plant.

22. The transgenic land plant of claim 1, wherein the transgenic land plant is a C4 plant.

23. The transgenic land plant of claim 1, wherein the transgenic land plant is a food crop plant selected from the group consisting of maize, wheat, oat, barley, soybean, millet, sorghum, potato, pulse, bean, tomato, and rice.

24. The transgenic land plant of claim 1, wherein the transgenic land plant is a forage crop plant selected from the group consisting of hay, alfalfa, and silage corn.

25. The transgenic land plant of claim 1, wherein the transgenic land plant is an oilseed crop plant selected from the group consisting of camelina, Brassica species (e.g. B. napus (canola), B. rapa, B. juncea, and B. carinata), crambe, soybean, sunflower, safflower, oil palm, flax, and cotton.


SEQUENCE LISTING (11 sequences)

SEQ ID NO: 1 - protein, 358 aa, Chlamydomonas reinhardtii
MSSDAMTINESLMEVEHTPAVHKRILDILPGISGGVARVMIGQPFDTIKVRLQVLGQGTALAAKLPPSEVYKDSMDCIRKMIKSEGPLSFYKGTVAPLVGNMVLLGIHFPVFSAVRKQLEGDDHYSNFSHANVLLSGAAAGAAGSLISAPVELVRTKMQMQRRAALAGTVAAGAAASAGAEEFYKGSLDCFKQVMSKHGIKGLYRGFTSTILRDMQGYAWFFLGYEATVNHFLQNAGPGVHTKADLNYLQVMAAGVVAGFGLWGSMFPIDTIKSKLQADSFAKPQYSSTMDCLKKVLASEGQAGLWRGFSAAMYRAIPVNAGIFLAVEGTRQGIKWYEENVEHIYGGVIGPATPTAAQ

SEQ ID NO: 2 - protein, 353 aa, Chlorella sorokiniana
MVARTINETLMEVEHTPPVHKRVLDVLPGVSGGVTRVLVGQPFDTIKTRLQVMGQGTALAKMLPPSDVYINSSDCLKKMVRNEGALSLYRGVVAPLLGNMVLLGIHFPTFSNTRKYLESVDATPAGEFPYWKVLAAGGAAGLAGSFISCPSEHIRTKMQLQRRAALAAQMGLKAQGLETYKGSWDCAVQILRNHGIKGLYRGMTSTVLRDIQGYAWFFLCYEATLHALAGPAHTRSELDYKHVLGAGVMAGFGLWGSMFPIDTIKSKMQGDSLSNPQYRNTLDCLRQSVAVEGFGGLFRGFGAAMYRAIPVNAGIFLAVEGTRQLLNKYEGYIDEKLGISVPASAATVPAPAQ

SEQ ID NO: 3 - protein, 306 aa, Chlorella-like algae
MPHNETTPAALPFYKTFAASAAAACTGEVATIPMDTVKVRLQVQGASGAPAKYKGTLGTLAKVAREEGVASLYKGLVPGLHRQILLGGVRIATYDPIRDFYGRLMKEEAGHTSIPTKIAAALTAGTFGVLVGNPTDVLKVRMQAQGKLPAGTPSRYPSAMAAYGMIVRQEGVKALWTGTTPNIARNSVVNAAELATYDQIKQLLMASFGFHDNVYCHLSASLCAGFLAVAAGSPFDVIKSRAMALSATGGYQGVGHVVMQTMRNEGLLAFWSGFSANFLRLGSWNIAMFLTLEKLRHLMGAPSAKH

SEQ ID NO: 4 - protein, 323 aa, Chlorella-like algae
MQEIQMPAVPAPPTLAAPQPASGFVRFAKDSFAGTVGGIAVTMVGHPFDTVKVRLQTQPSVNPIYNGAIDCVKKTLQWEGVPGLYKGVTSPLAGQMFFRATLFSAFGASKRWLGTNADGTTRDLTTADYYKAGFITGAAAAFTEAPIDFYKSQIQVQMVRAKADPTYKAPYTSVGECIKATVRYSGFKAPFQGLSATLLRNAPANAIYLGSFEVLKQQASKYYGCAPKDLSAPVVMAAGGTGGILYWLAIFPVDVIKSAMMTDSIDPAQRKYPTIPSTAKALWAEGGLSRFYRGFSPCIMRAAPANAVMLFTVDRVSHLLSDH

SEQ ID NO: 5 - protein, 323 aa, Chlorella-like algae
MTAGKSGLHPAADYVAGAIAGSANIALGFPADTVKVRLQNRLNPYNGAWHCATSMLRNEGARSLYRGMSPQLVGGAVETGVNYAVYQAMLGLTQGPRLALPEAAAVPLSAAAAGAVLSVVLSPAELVKCRLQLGGTERYHSYRGPVDCLRQTVQQEGLRGLMRGLSGTMAREIPGNAIYFSTYRLLRYWVSGGDPAATAAAASGATVAAASQPRSLLAFLVDSASAVVCGGLAGMVMWAAVLPLDVAKTRIQTAYPGSYQDVGVARQLHMVYREGGIQALYAGLSPTLARAFPANAAQWLAWELCMQQMQQWGGGGGRGGSST

SEQ ID NO: 6 - protein, 303 aa, Chlorella-like algae
MRTGVAVDLASGTAAGAAQLLVGHPFDTIKVNMQVGSADTTAMGAARRIVGTHGPLGMYRGLAAPLATVAAFNAVLFSSWGATERMLSPDGGCCPLTVGQAMLAGGLAGVPVSLLATPTELLKCRLQAQGGARPPPGMVYSLADIRAGRALFNGPLDVLRHVVRHEGGWLGAYRGLGATLLREVPGNAAYFGVYEGCKYGLARWQCIPTSELGPASLMTAGGVGGAAFWIVTYPFDVVKSRLQTQNIHALDRYHGTWDCMTRLYSAQGWQALWRGFGPCMARSVPANAVAFLAFEQVRAALSH

SEQ ID NO: 7 - protein, 328 aa, Chondrus crispus
MPSTTPLVDATSPAAATPDASATAVPAPVSIAAAAGPVYPPYAHALAGAGGGLATVTLLHPLDTLRTRLQSVERRAVLARRGDAVRAFKEILVREGAPALYRGVVPAAFGSVLSWACYFHWFQRARTIVKPAITHETGSHLLAGTIAGLMTSFATNPIWVVKVRLQLQRTGKSVAPGFKPYSGFFDGLKSITREEGVRGLYRGIGPSVWLVSHGAVQFTMYERFKERLRQDADPQSGTTVFHSLIASTGSKLVASLATYPLQVARTRMQERFADGRRYGNFHTAFMYIFRTEGIRGLYRGLSANVIRVTPQAAVTFITYEQILKLCAN

SEQ ID NO: 8 - protein, 233 aa, Chondrus crispus
VSREGAAGLYAGIQAPLPFVAVFNATLFAANSTMRKVVGKGRPDDDLSIAQIGLAGAGAGAAVSFVACPTELVKCRLQAQPGAFNGAIDCTRQVVANRGMGGLFTGMGATMVREMPGNALMFMTYNATMRALCSPGQATKDLSASQLMFAGGMAGLAFWMPCYPIDFAKTLIQTDSETNPRYRGLLDCMRKTVKAEGVGGLYKGIGPCLARAVPANAVTFLIYQWTLQLLGHS

SEQ ID NO: 9 - protein, 194 aa, Chondrus crispus
MGRPDDDLSIAQIGLAGAGAGPAVSFVACPTELIKCRLQAQPGAFNGAIDCTRQVVANRGMGGLFTGMGATMVREMPGNALMFMTYNATMRALCSPGQATKDLSASQLMFAGGMACLAFWMPCYPIDFAKTLIQTDSETNPRYRGLLDCMRKTVKAEGVGGLYKGIGPCLARAVPANAVTFLIDQCTLQLLGHS

SEQ ID NO: 10 - DNA, 11031 bp, Artificial Sequence
  pMBXO85
tcgagtttctccataataatgtgtgagtagttcccagataagggaattagggttcctatagggtttcgctcatgtgttgagcatataagaaacccttagtatgtatttgtatttgtaaaatacttctatcaataaaatttctaattcctaaaaccaaaatccagtactaaaatccagatcccccgaattaattcggcgttaattcagtacattaaaaacgtccgcaatgtgttattaagttgtctaagcgtcaatttgtttacaccacaatatatcctgccaccagccagccaacagctccccgaccggcagctcggcacaaaatcaccactcgatacaggcagcccatcagtccgggacggcgtcagcgggagagccgttgtaaggcggcagactttgctcatgttaccgatgctattcggaagaacggcaactaagctgccgggtttgaaacacggatgatctcgcggagggtagcatgttgattgtaacgatgacagagcgttgctgcctgtgatcaccgcggtttcaaaatcggctccgtcgatactatgttatacgccaactttgaaaacaactttgaaaaagctgttttctggtatttaaggttttagaatgcaaggaacagtgaattggagttcgtcttgttataattagcttcttggggtatctttaaatactgtagaaaagaggaaggaaataataaatggctaaaatgagaatatcaccggaattgaaaaaactgatcgaaaaataccgctgcgtaaaagatacggaaggaatgtctcctgctaaggtatataagctggtgggagaaaatgaaaacctatatttaaaaatgacggacagccggtataaagggaccacctatgatgtggaacgggaaaaggacatgatgctatggctggaaggaaagctgcctgttccaaaggtcctgcactttgaacggcatgatggctggagcaatctgctcatgagtgaggccgatggcgtcctttgctcggaagagtatgaagatgaacaaagccctgaaaagattatcgagctgtatgcggagtgcatcaggctctttcactccatcgacatatcggattgtccctatacgaatagcttagacagccgcttagccgaattggattacttactgaataacgatctggccgatgtggattgcgaaaactgggaagaagacactccatttaaagatccgcgcgagctgtatgattttttaaagacggaaaagcccgaagaggaacttgtcttttcccacggcgacctgggagacagcaacatctttgtgaaagatggcaaagtaagtggctttattgatcttgggagaagcggcagggcggacaagtggtatgacattgccttctgcgtccggtcgatcagggaggatatcggggaagaacagtatgtcgagctattttttgacttactggggatcaagcctgattgggagaaaataaaatattatattttactggatgaattgttttagtacctagaatgcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagggtgccttgatgtgggcgccggcggtcgagtggcgacggcgcggcttgtccgcgccctggtagattgcctggccgtaggccagccatttttgagcggccagcggccgcgataggccgacgcgaagcggcggggcgtagggagcgcagcgaccgaagggtaggcgctttttgcagctcttcggctgtgcgctggccagacagttatgcacaggccaggcgggttttaagagttttaataagttttaaagagttttaggcggaaaaatcgccttttttctcttttatatcagtcacttacatgtgtgaccggttcccaatgtacggctttgggttcccaatgtacgggttccggttcccaatgtacggctttgggttcccaatgtacgtgctatccacaggaaagagaccttttcgacctttttcccctgctagggcaatttgccctagcatctgctccgtacattaggaaccggcggatgcttcgccctcgatcaggttgcggtagcgcatgactaggatcgggccagcctgccccgcctcctccttcaaatcgtactccggcaggtcatttgacccgatcagcttgcgcacggtgaaacagaacttcttgaactctccggcgctgccactgcgttcgtagatcgtcttgaacaaccatctggcttctgccttgcctgcggcgcggcgtgccaggcggtagagaaaacggccgatgccgggatcgatcaaaaagtaatcggggtgaaccgtcagcacgtccgggttcttgccttctgtgatctcgcggtacatccaatcagctagctcgatctcgatgtactccggccgcccggtttcgctctttacgatcttgtagcggctaatcaaggcttcaccctcggataccgtcaccaggcggccgttcttggccttcttcgtacgctgcatggcaacgtgcgtggtgtttaaccgaatgcaggtttctaccaggtcgtctttctgctttccgccatcggctcgccggcagaacttgagtacgtccgcaacgtgtggacggaacacgcggccgggcttgtctcccttcccttcccggtatcggttcatggattcggttagatgggaaaccgccatcagtaccaggtcgtaatcccacacactggccatgccggccggccctgcggaaacctctacgtgcccgtctggaagctcgtagcggatcacctcgccagctcgtcggtcacgcttcgacagacggaaaacggccacgtccatgatgctgcgactatcgcgggtgcccacgtcatagagcatcggaacgaaaaaatctggttgctcgtcgcccttgggcggcttcctaatcgacggcgcaccggctgccggcggttgccgggattctttgcggattcgatcagcggccgcttgccacgattcaccggggcgtgcttctgcctcgatgcgttgccgctgggcggcctgcgcggccttcaacttctccaccaggtcatcacccagcgccgcgccgatttgtaccgggccggatggtttgcgaccgtcacgccgattcctcgggcttgggggttccagtgccattgcagggccggcagacaacccagccgcttacgcctggccaaccgcccgttcctccacacatggggcattccacggcgtcggtgcctggttgttcttgattttccatgccgcctcctttagccgctaaaattcatctactcatttattcatttgctcatttactctggtagctgcgcgatgtattcagatagcagctcggtaatggtcttgccttggcgtaccgcgtacatcttcagcttggtgtgatcctccgccggcaactgaaagttgacccgcttcatggctggcgtgtctgccaggctggccaacgttgcagccttgctgctgcgtgcgctcggacggccggcacttagcgtgtttgtgcttttgctcattttctctttacctcattaactcaaatgagttttgatttaatttcagcggccagcgcctggacctcgcgggcagcgtcgccctcgggttctgattcaagaacggttgtgccggcggcggcagtgcctgggtagctcacgcgctgcgtgatacgggactcaagaatgggcagctcgtacccggccagcgcctcggcaacctcaccgccgatgcgcgtgcctttgatcgcccgcgacacgacaaaggccgcttgtagccttccatccgtgacctcaatgcgctgcttaaccagctccaccaggtcggcggtggcccatatgtcgtaagggcttggctgcaccggaatcagcacgaagtcggctgccttgatcgcggacacagccaagtccgccgcctggggcgctccgtcgatcactacgaagtcgcgccggccgatggccttcacgtcgcggtcaatcgtcgggcggtcgatgccgacaacggttagcggttgatcttcccgcacggccgcccaatcgcgggcactgccctggggatcggaatcgactaacagaacatcggccccggcgagttgcagggcgcgggctagatgggttgcgatggtcgtcttgcctgacccgcctttctggttaagtacagcgataaccttcatgcgttccccttgcgtatttgtttatttactcatcgcatcatatacgcagcgaccgcatgacgcaagctgttttactcaaatacacatcacctttttagacggcggcgctcggtttcttcagcggccaagctggccggccaggccgccagcttggcatcagacaaaccggccaggatttcatgcagccgcacggttgagacgtgcgcgggcggctcgaacacgtacccggccgcgatcatctccgcctcgatctcttcggtaatgaaaaacggttcgtcctggccgtcctggtgcggtttcatgcttgttcctcttggcgttcattctcggcggccgccagggcgtcggcctcggtcaatgcgtcctcacggaaggcaccgcgccgcctggcctcggtgggcgtcacttcctcgctgcgctcaagtgcgcggtacagggtcgagcgatgcacgccaagcagtgcagccgcctctttcacggtgcggccttcctggtcgatcagctcgcgggcgtgcgcgatctgtgccggggtgagggtagggcgggggccaaacttcacgcctcgggccttggcggcctcgcgcccgctccgggtgcggtcgatgattagggaacgctcgaactcggcaatgccggcgaacacggtcaacaccatgcggccggccggcgtggtggtgtcggcccacggctctgccaggctacgcaggcccgcgccggcctcctggatgcgctcggcaatgtccagtaggtcgcgggtgctgcgggccaggcggtctagcctggtcactgtcacaacgtcgccagggcgtaggtggtcaagcatcctggccagctccgggcggtcgcgcctggtgccggtgatcttctcggaaaacagcttggtgcagccggccgcgtgcagttcggcccgttggttggtcaagtcctggtcgtcggtgctgacgcgggcatagcccagcaggccagcggcggcgctcttgttcatggcgtaatgtctccggttctagtcgcaagtattctactttatgcgactaaaacacgcgacaagaaaacgccaggaaaagggcagggcggcagcctgtcgcgtaacttaggacttgtgcgacatgtcgttttcagaagacggctgcactgaacgtcagaagccgactgcactatagcagcggaggggttggatcaaagtactttgatcccgaggggaaccctgtggttggcatgcacatacaaatggacgaacggataaaccttttcacgcccttttaaatatccgttattctaataaacgctcttttctcttaggtttacccgccaatatatcctgtcaaacactgatagtttaaactgaaggcgggaaacgacaatctgatccaagctcaagctgctctagcattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccaagcttcaatcccacaaaaatctgagcttaacagcacagttgctcctctcagagcagaatcgggtattcaacaccctcatatcaactactacgttgtgtataacggtccacatgccggtatatacgatgactggggttgtacaaaggcggcaacaaacggcgttcccggagttgcacacaagaaatttgccactattacagaggcaagagcagcagctgacgcgtacacaacaagtcagcaaacagacaggttgaacttcatccccaaaggagaagctcaactcaagcccaagagctttgctaaggccctaacaagcccaccaaagcaaaaagcccactggctcacgctaggaaccaaaaggcccagcagtgatccagccccaaaagagatctcctttgccccggagattacaatggacgatttcctctatctttacgatctaggaaggaagttcgaaggtgaaggtgacgacactatgttcaccactgataatgagaaggttagcctcttcaatttcagaaagaatgctgacccacagatggttagagaggcctacgcagcaggtctcatcaagacgatctacccgagtaacaatctccaggagatcaaataccttcccaagaaggttaaagatgcagtcaaaagattcaggactaattgcatcaagaacacagagaaagacatatttctcaagatcagaagtactattccagtatggacgattcaaggcttgcttcataaaccaaggcaagtaatagagattggagtctctaaaaaggtagttcctactgaatctaaggccatgcatggagtctaagattcaaatcgaggatctaacagaactcgccgtgaagactggcgaacagttcatacagagtcttttacgactcaatgacaagaagaaaatcttcgtcaacatggtggagcacgacactctggtctactccaaaaatgtcaaagatacagtctcagaagaccaaagggctattgagacttttcaacaaaggataatttcgggaaacctcctcggattccattgcccagctatctgtcacttcatcgaaaggacagtagaaaaggaaggtggctcctacaaatgccatcattgcgataaaggaaaggctatcattcaagatctctctgccgacagtggtcccaaagatggacccccacccacgaggagcatcgtggaaaaagaagacgttccaaccacgtcttcaaagcaagtggattgatgtgacatctccactgacgtaagggatgacgcacaatcccactatccttcgcaagacccttcctctatataaggaagttcatttcatttggagaggacacgaaaatggttgcaagaaccattaacgagactcttatggaagtggaacatacaccgccagtgcacaagagagtcctagatgttttacctggggtttcaggaggcgtgactagggtgttagttggacagccattcgatacaattaaaactagattacaggtcatgggtcagggtacggcattggctaaaatgcttccaccttctgatgtgtatattaactcttccgactgtttgaaaaaaatggtacgtaacgaaggtgctttgtctctgtaccgtggtgtggttgcgcctcttttaggaaatatggtgctgcttggaattcatttccctaccttttcgaacacaagaaagtaccttgagtctgttgacgctacaccagcgggcgaatttccttactggaaggtgttagctgcaggtggcgcagcagggctcgcaggatctttcatatcttgtccttcggagcacatacgtactaagatgcaattacagcgtagagcggcccttgccgcacagatgggtttgaaggctcaagggctggagacttacaagggatcctgggattgtgctgtgcaaattttgaggaaccacggcataaagggactctacaggggaatgaccagtacggtgttaagggatatacaaggctacgcatggttcttcctctgttacgaagctacattgcacgccttagcaggaccagctcatacacgttctgaattggattacaagcacgtcttgggagctggagttatggcagggttcggtctctgggggtccatgtttccaatcgacacaataaaatccaaaatgcagggtgattctcttagtaacccccagtatcgtaacacgctggattgtctccgacagagtgtagcagttgaaggtttcgggggcctttttagagggttcggcgctgcaatgtacagagctattccagttaatgcaggtatatttcttgccgtagagggaacaagacaacttctaaacaagtacgagggttacattgatgagaagttgggtatttctgttcctgcttctgcggcgacagttcctgctccggcgcaatagggatccatttaaatgtttctccataataatgtgtgagtagttcccagataagggaattagggttcctatagggtttcgctcatgtgttgagcatataagaaacccttagtatgtatttgtatttgtaaaatacttctatcaataaaatttctaattcctaaaaccaaaatccagtactaaaatccagatcccccgaattaattcggcgttaattcagactagtcgtcaaagggcgacaccccctaattagcccaattcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattggctagagcagcttgccaacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgataacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagaccttcctctatataaggaagttcatttcatttggagaggacacgctgaaatcaccagtctctctctacaaatctatctctctcgagtctaccatgagcccagaacgacgcccggccgacatccgccgtgccaccgaggcggacatgccggcggtctgcaccatcgtcaaccactacatcgagacaagcacggtcaacttccgtaccgagccgcaggaaccgcaggagtggacggacgacctcgtccgtctgcgggagcgctatccctggctcgtcgccgaggtggacggcgaggtcgccggcatcgcctacgcgggcccctggaaggcacgcaacgcctacgactggacggccgagtcgaccgtgtacgtctccccccgccaccagcggacgggactgggctccacgctctacacccacctgctgaagtccctggaggcacagggcttcaagagcgtggtcgctgtcatcgggctgcccaacgacccgagcgtgcgcatgcacgaggcgctcggatatgccccccgcggcatgctgcgggcggccggcttcaagcacgggaactggcatgacgtgggtttctggcagctggacttcagcctgccggtaccgccccgtccggtcctgcccgtcaccgagatttgac

SEQ ID NO: 11 - DNA, 10881 bp, Artificial Sequence
  pMBXO86
tcgagtttctccataataatgtgtgagtagttcccagataagggaattagggttcctatagggtttcgctcatgtgttgagcatataagaaacccttagtatgtatttgtatttgtaaaatacttctatcaataaaatttctaattcctaaaaccaaaatccagtactaaaatccagatcccccgaattaattcggcgttaattcagtacattaaaaacgtccgcaatgtgttattaagttgtctaagcgtcaatttgtttacaccacaatatatcctgccaccagccagccaacagctccccgaccggcagctcggcacaaaatcaccactcgatacaggcagcccatcagtccgggacggcgtcagcgggagagccgttgtaaggcggcagactttgctcatgttaccgatgctattcggaagaacggcaactaagctgccgggtttgaaacacggatgatctcgcggagggtagcatgttgattgtaacgatgacagagcgttgctgcctgtgatcaccgcggtttcaaaatcggctccgtcgatactatgttatacgccaactttgaaaacaactttgaaaaagctgttttctggtatttaaggttttagaatgcaaggaacagtgaattggagttcgtcttgttataattagcttcttggggtatctttaaatactgtagaaaagaggaaggaaataataaatggctaaaatgagaatatcaccggaattgaaaaaactgatcgaaaaataccgctgcgtaaaagatacggaaggaatgtctcctgctaaggtatataagctggtgggagaaaatgaaaacctatatttaaaaatgacggacagccggtataaagggaccacctatgatgtggaacgggaaaaggacatgatgctatggctggaaggaaagctgcctgttccaaaggtcctgcactttgaacggcatgatggctggagcaatctgctcatgagtgaggccgatggcgtcctttgctcggaagagtatgaagatgaacaaagccctgaaaagattatcgagctgtatgcggagtgcatcaggctctttcactccatcgacatatcggattgtccctatacgaatagcttagacagccgcttagccgaattggattacttactgaataacgatctggccgatgtggattgcgaaaactgggaagaagacactccatttaaagatccgcgcgagctgtatgattttttaaagacggaaaagcccgaagaggaacttgtcttttcccacggcgacctgggagacagcaacatctttgtgaaagatggcaaagtaagtggctttattgatcttgggagaagcggcagggcggacaagtggtatgacattgccttctgcgtccggtcgatcagggaggatatcggggaagaacagtatgtcgagctattttttgacttactggggatcaagcctgattgggagaaaataaaatattatattttactggatgaattgttttagtacctagaatgcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagggtgccttgatgtgggcgccggcggtcgagtggcgacggcgcggcttgtccgcgccctggtagattgcctggccgtaggccagccatttttgagcggccagcggccgcgataggccgacgcgaagcggcggggcgtagggagcgcagcgaccgaagggtaggcgctttttgcagctcttcggctgtgcgctggccagacagttatgcacaggccaggcgggttttaagagttttaataagttttaaagagttttaggcggaaaaatcgccttttttctcttttatatcagtcacttacatgtgtgaccggttcccaatgtacggctttgggttcccaatgtacgggttccggttcccaatgtacggctttgggttcccaatgtacgtgctatccacaggaaagagaccttttcgacctttttcccctgctagggcaatttgccctagcatctgctccgtacattaggaaccggcggatgcttcgccctcgatcaggttgcggtagcgcatgactaggatcgggccagcctgccccgcctcctccttcaaatcgtactccggcaggtcatttgacccgatcagcttgcgcacggtgaaacagaacttcttgaactctccggcgctgccactgcgttcgtagatcgtcttgaacaaccatctggcttctgccttgcctgcggcgcggcgtgccaggcggtagagaaaacggccgatgccgggatcgatcaaaaagtaatcggggtgaaccgtcagcacgtccgggttcttgccttctgtgatctcgcggtacatccaatcagctagctcgatctcgatgtactccggccgcccggtttcgctctttacgatcttgtagcggctaatcaaggcttcaccctcggataccgtcaccaggcggccgttcttggccttcttcgtacgctgcatggcaacgtgcgtggtgtttaaccgaatgcaggtttctaccaggtcgtctttctgctttccgccatcggctcgccggcagaacttgagtacgtccgcaacgtgtggacggaacacgcggccgggcttgtctcccttcccttcccggtatcggttcatggattcggttagatgggaaaccgccatcagtaccaggtcgtaatcccacacactggccatgccggccggccctgcggaaacctctacgtgcccgtctggaagctcgtagcggatcacctcgccagctcgtcggtcacgcttcgacagacggaaaacggccacgtccatgatgctgcgactatcgcgggtgcccacgtcatagagcatcggaacgaaaaaatctggttgctcgtcgcccttgggcggcttcctaatcgacggcgcaccggctgccggcggttgccgggattctttgcggattcgatcagcggccgcttgccacgattcaccggggcgtgcttctgcctcgatgcgttgccgctgggcggcctgcgcggccttcaacttctccaccaggtcatcacccagcgccgcgccgatttgtaccgggccggatggtttgcgaccgtcacgccgattcctcgggcttgggggttccagtgccattgcagggccggcagacaacccagccgcttacgcctggccaaccgcccgttcctccacacatggggcattccacggcgtcggtgcctggttgttcttgattttccatgccgcctcctttagccgctaaaattcatctactcatttattcatttgctcatttactctggtagctgcgcgatgtattcagatagcagctcggtaatggtcttgccttggcgtaccgcgtacatcttcagcttggtgtgatcctccgccggcaactgaaagttgacccgcttcatggctggcgtgtctgccaggctggccaacgttgcagccttgctgctgcgtgcgctcggacggccggcacttagcgtgtttgtgcttttgctcattttctctttacctcattaactcaaatgagttttgatttaatttcagcggccagcgcctggacctcgcgggcagcgtcgccctcgggttctgattcaagaacggttgtgccggcggcggcagtgcctgggtagctcacgcgctgcgtgatacgggactcaagaatgggcagctcgtacccggccagcgcctcggcaacctcaccgccgatgcgcgtgcctttgatcgcccgcgacacgacaaaggccgcttgtagccttccatccgtgacctcaatgcgctgcttaaccagctccaccaggtcggcggtggcccatatgtcgtaagggcttggctgcaccggaatcagcacgaagtcggctgccttgatcgcggacacagccaagtccgccgcctggggcgctccgtcgatcactacgaagtcgcgccggccgatggccttcacgtcgcggtcaatcgtcgggcggtcgatgccgacaacggttagcggttgatcttcccgcacggccgcccaatcgcgggcactgccctggggatcggaatcgactaacagaacatcggccccggcgagttgcagggcgcgggctagatgggttgcgatggtcgtcttgcctgacccgcctttctggttaagtacagcgataaccttcatgcgttccccttgcgtatttgtttatttactcatcgcatcatatacgcagcgaccgcatgacgcaagctgttttactcaaatacacatcacctttttagacggcggcgctcggtttcttcagcggccaagctggccggccaggccgccagcttggcatcagacaaaccggccaggatttcatgcagccgcacggttgagacgtgcgcgggcggctcgaacacgtacccggccgcgatcatctccgcctcgatctcttcggtaatgaaaaacggttcgtcctggccgtcctggtgcggtttcatgcttgttcctcttggcgttcattctcggcggccgccagggcgtcggcctcggtcaatgcgtcctcacggaaggcaccgcgccgcctggcctcggtgggcgtcacttcctcgctgcgctcaagtgcgcggtacagggtcgagcgatgcacgccaagcagtgcagccgcctctttcacggtgcggccttcctggtcgatcagctcgcgggcgtgcgcgatctgtgccggggtgagggtagggcgggggccaaacttcacgcctcgggccttggcggcctcgcgcccgctccgggtgcggtcgatgattagggaacgctcgaactcggcaatgccggcgaacacggtcaacaccatgcggccggccggcgtggtggtgtcggcccacggctctgccaggctacgcaggcccgcgccggcctcctggatgcgctcggcaatgtccagtaggtcgcgggtgctgcgggccaggcggtctagcctggtcactgtcacaacgtcgccagggcgtaggtggtcaagcatcctggccagctccgggcggtcgcgcctggtgccggtgatcttctcggaaaacagcttggtgcagccggccgcgtgcagttcggcccgttggttggtcaagtcctggtcgtcggtgctgacgcgggcatagcccagcaggccagcggcggcgctcttgttcatggcgtaatgtctccggttctagtcgcaagtattctactttatgcgactaaaacacgcgacaagaaaacgccaggaaaagggcagggcggcagcctgtcgcgtaacttaggacttgtgcgacatgtcgttttcagaagacggctgcactgaacgtcagaagccgactgcactatagcagcggaggggttggatcaaagtactttgatcccgaggggaaccctgtggttggcatgcacatacaaatggacgaacggataaaccttttcacgcccttttaaatatccgttattctaataaacgctcttttctcttaggtttacccgccaatatatcctgtcaaacactgatagtttaaactgaaggcgggaaacgacaatctgatccaagctcaagctgctctagcattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccaagcttcaatcccacaaaaatctgagcttaacagcacagttgctcctctcagagcagaatcgggtattcaacaccctcatatcaactactacgttgtgtataacggtccacatgccggtatatacgatgactggggttgtacaaaggcggcaacaaacggcgttcccggagttgcacacaagaaatttgccactattacagaggcaagagcagcagctgacgcgtacacaacaagtcagcaaacagacaggttgaacttcatccccaaaggagaagctcaactcaagcccaagagctttgctaaggccctaacaagcccaccaaagcaaaaagcccactggctcacgctaggaaccaaaaggcccagcagtgatccagccccaaaagagatctcctttgccccggagattacaatggacgatttcctctatctttacgatctaggaaggaagttcgaaggtgaaggtgacgacactatgttcaccactgataatgagaaggttagcctcttcaatttcagaaagaatgctgacccacagatggttagagaggcctacgcagcaggtctcatcaagacgatctacccgagtaacaatctccaggagatcaaataccttcccaagaaggttaaagatgcagtcaaaagattcaggactaattgcatcaagaacacagagaaagacatatttctcaagatcagaagtactattccagtatggacgattcaaggcttgcttcataaaccaaggcaagtaatagagattggagtctctaaaaaggtagttcctactgaatctaaggccatgcatggagtctaagattcaaatcgaggatctaacagaactcgccgtgaagactggcgaacagttcatacagagtcttttacgactcaatgacaagaagaaaatcttcgtcaacatggtggagcacgacactctggtctactccaaaaatgtcaaagatacagtctcagaagaccaaagggctattgagacttttcaacaaaggataatttcgggaaacctcctcggattccattgcccagctatctgtcacttcatcgaaaggacagtagaaaaggaaggtggctcctacaaatgccatcattgcgataaaggaaaggctatcattcaagatctctctgccgacagtggtcccaaagatggacccccacccacgaggagcatcgtggaaaaagaagacgttccaaccacgtcttcaaagcaagtggattgatgtgacatctccactgacgtaagggatgacgcacaatcccactatccttcgcaagacccttcctctatataaggaagttcatttcatttggagaggacacgaaaatgcgtacgggagttgcagtcgatctagcttccggaacagcggcaggggctgcccaattactcgtcggtcacccttttgacactatcaaagtgaatatgcaagttggatctgccgatactactgcaatgggagctgctagaaggattgtgggaactcacggccctttaggaatgtatagaggactggctgctcctttggcgacggtagcagcttttaacgcagtcctgttcagtagctggggtgctactgagagaatgttgtcccccgacggtggatgttgtcctcttacggtgggtcaagctatgctcgcgggaggtttagctggtgtcccagtaagcttgcttgccaccccaacagaactactcaaatgtcgtcttcaagctcaaggtggcgctagacctccgccaggtatggtttattcccttgctgatattcgtgcaggtcgtgctctttttaatggtcctctcgacgttttaagacatgtggttaggcatgaagggggttggttgggcgcctacagaggacttggtgcgacactcctccgtgaagttccgggcaacgcggcttacttcggtgtatatgagggttgtaaatatggtttagctaggtggcaatgtatccctacttccgagctcggcccggccagccttatgacagctggaggtgttggtggagccgctttttggatcgttacctaccctttcgatgttgtgaagtctagactccaaacccagaacatacacgcgcttgatcgatatcacggaacctgggattgtatgacacgtctctactctgcccagggttggcaggcactgtggagagggtttggcccttgcatggctagatctgtccccgccaatgcggttgcatttctggcgtttgaacaagtacgagctgctttgtcccattgaggatccatttaaatgtttctccataataatgtgtgagtagttcccagataagggaattagggttcctatagggtttcgctcatgtgttgagcatataagaaacccttagtatgtatttgtatttgtaaaatacttctatcaataaaatttctaattcctaaaaccaaaatccagtactaaaatccagatcccccgaattaattcggcgttaattcagactagtcgtcaaagggcgacaccccctaattagcccaattcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattggctagagcagcttgccaacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgataacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagaccttcctctatataaggaagttcatttcatttggagaggacacgctgaaatcaccagtctctctctacaaatctatctctctcgagtctaccatgagcccagaacgacgcccggccgacatccgccgtgccaccgaggcggacatgccggcggtctgcaccatcgtcaaccactacatcgagacaagcacggtcaacttccgtaccgagccgcaggaaccgcaggagtggacggacgacctcgtccgtctgcgggagcgctatccctggctcgtcgccgaggtggacggcgaggtcgccggcatcgcctacgcgggcccctggaaggcacgcaacgcctacgactggacggccgagtcgaccgtgtacgtctccccccgccaccagcggacgggactgggctccacgctctacacccacctgctgaagtccctggaggcacagggcttcaagagcgtggtcgctgtcatcgggctgcccaacgacccgagcgtgcgcatgcacgaggcgctcggatatgccccccgcggcatgctgcgggcggccggcttcaagcacgggaactggcatgacgtgggtttctggcagctggacttcagcctgccggtaccgccccgtccggtcctgcccgtcaccgagatttgac